SEQUENCE LISTING (19 sequences)

SEQ ID NO: 1 - protein, 146 aa, synthetic construct
  REGION 1..146: note = gp41 variant (N637E/H643Y)
TLTVQARQLLSGIVQQQNNELRAIEAQQHLLQLTVWGTKQLQARELAVERYLKDQQLLGIWGASGKLIATTAVPWNASWSNKSLEQIWNNMTWMEWDREINEYTSLIYSLIEESQNQQEKNEQELLELDKWASLWNWFNITNWLWY

SEQ ID NO: 2 - protein, 256 aa, synthetic construct
  REGION 1..256: note = 6hel (N636D/H643Y,2mut)
QLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREINNYTSLIHSLIEESQNQQEKNEQELLE

SEQ ID NO: 3 - protein, 256 aa, synthetic construct
  REGION 1..256: note = 6hel (N636D/H643Y,3mut)
QLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLE

SEQ ID NO: 4 - protein, 269 aa, synthetic construct
  REGION 1..269: note = 6hel-N(543-581)x3/C(625-662)x3-Srt wild type
MQLLSGIVQQQNNLLRAIEAQQHLLQLTVWGIKQLQARILGGSGGHTTWMEWDREINNYTSLIHSLIEESQNQQEKNEQELLEGSSGGQLLSGIVQQQNNLLRAIEAQQHLLQLTVWGIKQLQARILGGSGGHTTWMEWDREINNYTSLIHSLIEESQNQQEKNEQELLEGSSGGQLLSGIVQQQNNLLRAIEAQQHLLQLTVWGIKQLQARILGGRGGHTTWMEWDREINNYTSLIHSLIEESQNQQEKNEQELLGGLPETGHHHHHH

SEQ ID NO: 5 - protein, 275 aa, synthetic construct
  REGION 1..275: note = 6hel-N(543-581)x3/C(625-662,N636D/H643Y)x2/C(625-66 2)x1-Srt
MQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREINNYTSLIHSLIEESQNQQEKNEQELLEGGGLPETGSGGGHHHHHH

SEQ ID NO: 6 - protein, 271 aa, synthetic construct
  REGION 1..271: note = 6hel-(N(543-581)x3/C(625-662,N636D/H643Y)x3-Q-tag
MQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGGGYRYRQGGGHHH

SEQ ID NO: 7 - protein, 547 aa, synthetic construct
  REGION 1..547: note = EcSlyD-EcSlyD-gp41(536-681;N637E/H643Y)-Q-tag
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGSGGGSGGGTLTVQARQLLSGIVQQQNNELRAIEAQQHLLQLTVWGTKQLQARELAVERYLKDQQLLGIWGASGKLIATTAVPWNASWSNKSLEQIWNNMTWMEWDREINEYTSLIYSLIEESQNQQEKNEQELLELDKWASLWNWFNITNWLWYHGHDHDHDGGGSYRYRQGGGHHHHHH

SEQ ID NO: 8 - protein, 599 aa, synthetic construct
  REGION 1..599: note = EcSlyD-EcSlyD-Qtag-gp41(536-681;N637E/H643Y)
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGTLTVQARQLLSGIVQQQNNELRAIEAQQHLLQLTVWGTKQLQARELAVERYLKDQQLLGIWGASGKLIATTAVPWNASWSNKSLEQIWNNMTWMEWDREINEYTSLIYSLIEESQNQQEKNEQELLELDKWASLWNWFNITNWLWYHGHDHDHDHHHHHH

SEQ ID NO: 9 - protein, 267 aa, synthetic construct
  REGION 1..267: note = Six-Helix according to WO2001/044286
MQLLSGIVQQQNNLLRAIEAQQHLLQLTVWGIKQLQARILAGGSGGHTTWMEWDREINNYTSLIHSLIEESQNQQEKNEQELLEGSSGGQLLSGIVQQQNNLLRAIEAQQHLLQLTVWGIKQLQARILAGGSGGHTTWMEWDREINNYTSLIHSLIEESQNQQEKNEQELLEGSSGGQLLSGIVQQQNNLLRAIEAQQHLLQLTVWGIKQLQARILAGGRGGHTTWMEWDREINNYTSLIHSLIEESQNQQEKNEQELLGGHHHHHH

SEQ ID NO: 10 - protein, 146 aa, synthetic construct
  REGION 1..146: note = HIV gp41 according to WO03/0000877
TLTVQARQLLSGIVQQQNNELRAIEAQQHLLQLTVWGTKQLQARELAVERYLKDQQLLGIWGASGKLIATTAVPWNASWSNKSLEQIWNNMTWMEWDREINNYTSLIHSLIEESQNQQEKNEQELLELDKWASLWNWFNITNWLWY

SEQ ID NO: 11 - protein, 345 aa, synthetic construct
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSVVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGAYRAIRHIPRRIRQGLERILL

SEQ ID NO: 12 - protein, 5 aa, synthetic construct
  REGION 1..5: note = Sortase recognition sequence
LPETG

SEQ ID NO: 13 - protein, 5 aa, synthetic construct
  REGION 1..5: note = Transglutaminase recognition sequence
YRYRQ

SEQ ID NO: 14 - protein, 4 aa, synthetic construct
  REGION 1..4: note = Linker
GGGS

SEQ ID NO: 15 - protein, 15 aa, synthetic construct
  REGION 1..15: note = Linker
GGGSGGGSGGGSGGG

SEQ ID NO: 16 - protein, 4 aa, synthetic construct
  REGION 1..4: note = Linker
SGGG

SEQ ID NO: 17 - protein, 6 aa, synthetic construct
  REGION 1..6: note = His-Tag
HHHHHH

SEQ ID NO: 18 - protein, 39 aa, synthetic construct
  REGION 1..39: note = 6helNHR (543-581; 6-Helix N-heptad repeat)
QLLSGIVQQQNNLLRAIEAQQHLLQLTVWGIKQLQARIL

SEQ ID NO: 19 - protein, 38 aa, synthetic construct
  REGION 1..38: note = 6helCHR (625-662; 6-Helix C-heptad repeat)
HTTWMEWDREINNYTSLIHSLIEESQNQQEKNEQELLE

(19) United States
(12) Patent Application Publication
(10) Pub. No.: US 2025/0298022 A1
(43) Pub. Date: Sep. 25, 2025

(54) HIV GP41 VARIANTS FOR IMMUNODIAGNOSTIC ASSAYS

(71) Applicant: Roche Diagnostics Operations, Inc., Indianapolis, IN (US)

(72) Inventors: Juliane Benz, Weilheim (DE); Mara Boenitz-Dulat, Tutzing (DE); Mario Gloeck, Geretsried (DE); Peter Muench, Penzberg (DE); Daniela Poehlmann, Utting am Ammersee (DE); Alexander Riedel, Muenchen (DE)

(21) Appl. No.: 18/862,935

(22) PCT Filed: May 2, 2023
(86) PCT No.: PCT/EP2023/061443

§ 371 Date: Nov. 4, 2024

(30) Foreign Application Priority Data
May 3, 2022 (EP) 22171401.7

(51) Int. Cl. G01N 33/569; C07K 14/005

(52) U.S. Cl. CPC G01N 33/56988; C07K 14/005; C12N 2740/16022; C12N 2740/16051; G01N 2333/162; G01N 2469/20

(57) ABSTRACT

The invention relates to novel HIV gp41 antigen compositions that are suitable for detecting antibodies against HIV in an isolated biological sample providing high specificity immunoassay results. It further relates to methods detecting HIV antibodies, use of novel HIV gp41 antigen compositions in immunoassays as well as to reagent kits comprising novel HIV gp41 antigen compositions.

The present invention concerns HIV gp41 antigen compositions, and reagent kits comprising the same and methods of producing it. Also encompassed are methods of detecting anti-HIV antibodies in isolated samples using said HIV gp41 antigen compositions.

### BACKGROUND

The envelope proteins of human immunodeficiency virus (HIV) are essential for the cell infection process. In the first stages of an HIV infection the viral membrane undergoes a fusion process with the target cell membrane. Here, the viral envelope proteins are involved, i.e. gp41 and gp120, which both originate from the precursor protein gp160 that is proteolytically cleaved into these two fragments. The larger subunit gp120 is the surface-associated receptor binding subunit and gp41 forms the membrane spanning subunit which is involved in membrane fusion during virus entry into the target cell. As to the assumed binding mechanism from the virus with its target cell, contact of gp120/gp41 to the host cell membrane protein CD4 and other co-receptors triggers a series of conformational changes, leading to a formation of trimer-of-hairpins structure in gp41 (Root et al. Science 2001, 291, 884-888).

A patient infected with HIV usually develops antibodies against gp41 and other HIV proteins, so that for at least the past two decades gp41 has been a substantial ingredient for in vitro diagnostics immunoassays for detection of antibodies against HIV. Immunoassays applying the wild type sequence of HIV gp41 already show a high specificity. This means that samples containing HIV antibodies are usually correctly identified as positive.

However, there is still a substantial number of false positive samples which means that the assay result indicates to contain antibodies against HIV although in reality, the sample is negative and does not contain HIV antibodies. These false positives may become critical not only in a regular routine lab diagnostic setting as these results cause false alarms, intensive retesting and confirmatory testing procedures.

In addition, false positive results should particularly be avoided in a blood bank setting. Here, thousands of samples from blood donations are screened on a daily base on high throughput diagnostic analyzers, and a positive result means that the whole blood donation volume from a patient might be discarded.

Scholz et al. (J. Mol. Biol. 2005, 345, 1229-1241) describe gp41 polypeptide sequences from HIV-1 and the corresponding gp36 from HIV-2 that have been engineered in such a way that the aggregation-prone polypeptides can be expressed in a soluble form. However, these polypeptides, when used as an antigen in an in vitro diagnostic immunoassay for detection of HIV antibodies, do not completely avoid false positive results.

WO2001/044286 discloses an artificially designed Five-Helix protein with gp41 elements that can be used to inhibit HIV infection in human cells. This inhibitor comprises three stretches derived from the N-terminal helical domain from the gp41 and two stretches of the C-terminal helical domain from this molecule. However, this genetically engineered construct (also described by Root et al, supra) lacks many domains and many antigenic epitopes of the native molecule, and it especially does not contain the so-called loop motif, which is known to harbor particularly immunogenic epitopes. The Five-Helix protein folds into a stable structure and binds to a peptide that corresponds to the C-peptide region of HIV gp41 and thus inhibits HIV infection of human cells. It is also disclosed that the Five-Helix protein can be used as a drug-screening or antibody-screening tool. In addition, a Six-Helix protein, comprising gp41 sequences is disclosed. This Six-Helix protein, which comprises three N-helices and three C-Helices of HIV gp41, joined by linkers, can be used as a negative control in screening for drugs that inhibit membrane fusion.

While gp41 variants have been described in prior art widely, the publications are silent with regard to identification gp41 antigens that avoid false positive results in in vitro diagnostic immunoassays for detecting HIV antibodies.

The technical problem underlying the present invention may be seen in the provision of means and methods complying with the aforementioned needs, avoiding the problems identified as far as possible. The technical problem is solved by the embodiments characterized in the claims and described herein below.

### SUMMARY OF THE INVENTION

In a first aspect, the present invention relates to a composition suitable for detecting antibodies against HIV gp41 in an isolated sample, said composition comprising at least two individual HIV gp41 antigens, wherein a first HIV antigen comprises SEQ ID NO: 1 and wherein a second HIV gp41 antigen comprises at least one of SEQ ID NO: 2 or 3. In particular, said antigen comprises no further HIV specific amino acid sequences.

In a second aspect, the present invention relates to a method of producing a composition of HIV gp41 antigens, said method comprising for each of said antigens the steps of
a) culturing host cells, in particular E. coli cells, transformed with an expression vector comprising operably linked a recombinant DNA molecule encoding one of the antigens of the first aspect of the present invention,
b) expression of said antigen and
c) purification of said antigen and
d) admixing an HIV gp41 antigen comprising SEQ ID NO. 1 obtained by steps a) to c) with at least one HIV gp41 antigen comprising at least one of SEQ ID NO: 2 or 3 obtained by steps a) to c) to form a composition of HIV gp41 antigens.

In a third aspect, the present invention relates to a method for detecting antibodies specific for HIV in an isolated sample, wherein a composition according to the first aspect of the present invention, or an HIV gp41 antigen composition obtained by a method of the second aspect of the present invention is used as a capture reagent and/or as a binding partner for said anti-HIV antibodies.

In a fourth aspect, the present invention relates to a method for detecting antibodies specific for HIV in an isolated sample said method comprising
a) forming an immunoreaction mixture by admixing a body fluid sample with an HIV gp41 antigen composition of the first aspect of the present invention, or an HIV gp41 antigen composition obtained by the method of the second aspect of the present invention
b) maintaining said immunoreaction admixture for a time period sufficient for allowing antibodies present in the body fluid sample against said HIV gp41 antigen composition to immunoreact with an HIV gp41 antigen as part of said HIV gp41 antigen composition to form an immunoreaction product; and
c) detecting the presence and/or the concentration of any of said immunoreaction product.

In a fifth aspect, the present invention relates to a method of identifying if a patient has been exposed to an HIV infection in the past, comprising
a) forming an immunoreaction mixture by admixing a body fluid sample of the patient with a HIV gp41 antigen composition of the first aspect of the present invention or an HIV gp41 antigen composition obtained by the method of the second aspect of the present invention
b) maintaining said immunoreaction admixture for a time period sufficient for allowing antibodies present in the body fluid sample against said HIV gp41 antigen composition to immunoreact with an HIV gp41 antigen as part of said HIV gp41 antigen composition to form an immunoreaction product; and
c) detecting the presence and/or absence of any of said immunoreaction product, wherein the presence of an immunoreaction product indicates that the patient has been exposed to an HIV infection in the past.

In a sixth aspect, the present invention relates to a use of the HIV gp41 antigen composition of the first aspect of the present invention or of a HIV gp41 antigen composition obtained by the method of the second aspect of the present invention in a high throughput in vitro diagnostic test for the detection of anti-HIV antibodies.

In an seventh aspect, the present invention relates to a reagent kit for the detection of anti-HIV virus antibodies, comprising HIV gp41 antigen composition of the first aspect of the present invention or HIV gp41 antigen composition obtained by the method of the second aspect of the present invention.

### LIST OF FIGURES

FIG. 1: Sequence alignment of the wild type HIV gp41 (P03375, positions 512 to 868 shown as SEQ ID NO: 11) with the N-terminal (aa543-581; SEQ ID NO: 18) and the C-terminal (aa625-662; SEQ ID NO: 19) heptad repeats which are used in the 6hel (Six-Helix) constructs. Highlighted are the positions which are mutated to optimize the specificity of anti-HIV antigens (light grey: N636, dark grey N637, black H643). The bottom line of each of the upper three sections shows the position of gp41 of WO03/0000877 (SEQ ID NO: 10) in relation to the whole gp41 sequence.

FIG. 2: Amino acid sequence of the 6hel construct depicting the corresponding amino acid position in gp41 as well as the position in the heptad repeat (a to g). The N- and C-terminal heptad repeats are shown in light and dark grey, respectively. Positions marked with an x were mutated during optimization. The best positions are underlined.

FIG. 3a-f: Table listing all 242 designed HIV gp41 variants. Mutants which are highlighted in grey could either not be synthesized or expressed and thus could not be tested in an anti-HIV immunoassay.

FIG. 4: CD data of wild type recombinant 6hel (6hel_wt) antigen (SEQ ID NO: 4: black) as well as two mutated 6hel variants containing the N636D/H643Y mutations (SEQ ID NO: 3: light grey (6hel_N636D/H643Y,3mut) and SEQ ID NO: 2: dark grey (6hel_N636D/H643Y,2mut)). The CD spectra of all three antigen variants show the characteristic two minima of α-helices at 208 nm and 222 nm in the far-UV range strongly indicating that the introductions of points mutations in the 6hel construct does not interfere with the protein folding and thus does not harm the immune reactivity of the mutated antigens.

FIG. 5: HPLC analysis of the 6hel_wt antigen (SEQ ID NO: 4, A) in comparison with two different 6hel mutated antigens; B): SEQ ID NO: 2 (6hel_N636D/H643Y,2mut); C) SEQ ID NO: 3 (6hel_N636D/H643Y,3mut). The analysis was either done on a Superdex200 10/300 (A) or on a Superdex 5/150 (Band C) column. For quality control as well as for estimation of the molecular weight a HPLC standard (grey) is plotted together with the 6hel antigens (black) in the chromatograms. The HPLC-chromatograms strongly indicate that the introductions of point mutations in the 6hel antigen does not interfere with the protein folding and thus does not harm the immune reactivity of the mutated antigens.

FIG. 6: Performance of the improved anti-HIV module (AHIVII) comprising antigens with the SEQ ID NO: 1, 2 and 3 compared to the standard AHIV module of the Elecsys HIV Duo assay (AHIVI), comprising only antigens with the SEQ ID NO: 10. A detailed analysis is shown in the Examples section.

FIG. 7: Performance of the improved HIV Duo II assay comprising antigens with the SEQ ID NO: 1, 2 and 3 compared to the standard Elecsys HIV Duo assay comprising only antigens with the SEQ ID NO: 10. A) Shows the number of negative samples that are analyzed within an independent, external study as well as the number of false positive results within this study and the resulting specificity of the HIV Duo I assay compared to the HIV Duo II assay. B) Shows a detailed extraction of all false negative samples that interfere with the different gp41 and 6hel antigens used either in HIV Duo I (SEQ ID NO: 10) or HIV Duo II (SEQ ID NO: 1, 2 and 3). All samples with COI>0.8 are considered positive and highlighted in grey. For more details see the Examples section.

FIG. 8: Specificity and sensitivity of different combinations of gp41 and 6hel antigens. A) Specificity of an HIV immunoassay comprising antigens with SEQ ID NO: 10, SEQ ID NO: 1 and 2 or SEQ ID NO: 1 and 3. B) Sensitivity of the same antigen combinations was assessed using 10 different seroconversion samples that are close to the cutoff with the COIs going from light grey to dark grey. For more details see the Examples section.

List of Sequences

SEQ ID NO: 1: gp41 variant (N637E/H643Y) mutations are printed in bold and
underlined
TLTVQARQLL SGIVQQQNNE LRAIEAQQHL LQLTVWGTKQ LQARELAVER YLKDQQLLGI
WGASGKLIAT TAVPWNASWS NKSLEQIWNN MTWMEWDREI NEYTSLIYSL IEESQNQQEK
NEQELLELDK WASLWNWENI TNWLWY
SEQ ID NO: 2: 6hel (N636D/H643Y, 2mut), mutations are printed in bold and
underlined
QLLSGIVQQQ NNLLRAIEAQ QHLLQLTVWG IKQLQARILG GSGGHTTWME WDREIDNYTS
LIYSLIEESQ NQQEKNEQEL LEGSSGGQLL SGIVQQQNNL LRAIEAQQHL LQLTVWGIKQ
LQARILGGSG GHTTWMEWDR EIDNYTSLIY SLIEESQNQQ EKNEQELLEG SSGGQLLSGI
VQQQNNLLRA IEAQQHLLQL TVWGIKQLQA RILGGSGGHT TWMEWDREIN NYTSLIHSLI
EESQNQQEKN EQELLE
SEQ ID NO: 3: 6hel (N636D/H643Y, 3mut), mutations are printed in bold and
underlined
QLLSGIVQQQ NNLLRAIEAQ QHLLQLTVWG IKQLQARILG GSGGHTTWME WDREIDNYTS
LIYSLIEESQ NQQEKNEQEL LEGSSGGQLL SGIVQQQNNL LRAIEAQQHL LQLTVWGIKQ
LQARILGGSG GHTTWMEWDR EIDNYTSLIY SLIEESQNQQ EKNEQELLEG SSGGQLLSGI
VQQQNNLLRA IEAQQHLLQL TVWGIKQLQA RILGGSGGHT TWMEWDREID NYTSLIYSLI
EESQNQQEKN EQELLE
SEQ ID NO: 4: 6hel-N(543-581)x3/C(625-662)x3-Srt wild type
MQLLSGIVQQ QNNLLRAIEA QQHLLQLTVW GIKQLQARIL GGSGGHTTWM EWDREINNYT
SLIHSLIEES QNQQEKNEQE LLEGSSGGQL LSGIVQQQNN LLRAIEAQQH LLQLTVWGIK
QLQARILGGS GGHITWMEWD REINNYTSLI HSLIEESQNQ QEKNEQELLE GSSGGQLLSG
IVQQQNNLLR AIEAQQHLLQ LTVWGIKQLQ ARILGGRGGH TTWMEWDREI NNYTSLIHSL
IEESQNQQEK NEQELLGGLP ETGHHHHHH
SEQ ID NO: 5: 6hel-N(543-581)x3/C(625-662, N636D/H643Y)x2/C(625-662)x1-
Srt, mutations are printed in bold and underlined
MQLLSGIVQQ QNNLLRAIEA QQHLLQLTVW GIKQLQARIL GGSGGHTTWM EWDREIDNYT
SLIYSLIEES QNQQEKNEQE LLEGSSGGQL LSGIVQQQNN LLRAIEAQQH LLQLTVWGIK
QLQARILGGS GGHTTWMEWD REIDNYTSLI YSLIEESQNQ QEKNEQELLE GSSGGQLLSG
IVQQQNNLLR AIEAQQHLLQ LTVWGIKQLQ ARILGGSGGH TTWMEWDREI NNYTSLIHSL
IEESQNQQEK NEQELLEGGG LPETGSGGGH HHHHH
SEQ ID NO: 6: 6hel-(N(543-581)x3/C(625-662, N636D/H643Y)x3-Q-tag,
mutations are printed in bold and underlined
MQLLSGIVQQ QNNLLRAIEA QQHLLQLTVW GIKQLQARIL GGSGGHTTWM EWDREIDNYT
SLIYSLIEES QNQQEKNEQE LLEGSSGGQL LSGIVQQQNN LLRAIEAQQH LLQLTVWGIK
QLQARILGGS GGHTTWMEWD REIDNYTSLI YSLIEESQNQ QEKNEQELLE GSSGGQLLSG
IVQQQNNLLR AIEAQQHLLQ LTVWGIKQLQ ARILGGSGGH TTWMEWDREI DNYTSLIYSL
IEESQNQQEK NEQELLEGGG YRYRQGGGHH HHHH
SEQ ID NO: 7: EcSlyD-EcSlyD-gp41(536-681; N637E/H643Y)-Q-tag, mutations
are printed in bold and underlined
MKVAKDLVVS LAYQVRTEDG VLVDESPVSA PLDYLHGHGS LISGLETALE GHEVGDKEDV
AVGANDAYGQ YDENLVQRVP KDVFMGVDEL QVGMRFLAET DQGPVPVEIT AVEDDHVVVD
GNHMLAGQNL KFNVEVVAIR EATEEELAHG HVHGAHDHHH DHDHDGGGSG GGSGGGSGGG
SGGGSGGGKV AKDLVVSLAY QVRTEDGVLV DESPVSAPLD YLHGHGSLIS GLETALEGHE
VGDKFDVAVG ANDAYGQYDE NLVQRVPKDV FMGVDELQVG MRFLAETDQG PVPVEITAVE
DDHVVVDGNH MLAGQNLKFN VEVVAIREAT EEELAHGHVH GAHDHHHDHD HDGGGSGGGS
GGGSGGGSGG GSGGGTLTVQ ARQLLSGIVQ QQNNELRAIE AQQHLLQLTV WGTKQLQARE
LAVERYLKDQ QLLGIWGASG KLIATTAVPW NASWSNKSLE QIWNNMTWME WDREINEYTS
LIYSLIEESQ NQQEKNEQEL LELDKWASLW NWFNITNWLW YHGHDHDHDG GGSYRYRQGG
GHHHHHH
SEQ ID NO: 8: EcSlyD-EcSlyD-Qtag-gp41(536-681; N637E/H643Y), mutations
are printed in bold and underlined
MKVAKDLVVS LAYQVRTEDG VLVDESPVSA PLDYLHGHGS LISGLETALE GHEVGDKEDV
AVGANDAYGQ YDENLVQRVP KDVFMGVDEL QVGMRFLAET DQGPVPVEIT AVEDDHVVVD
GNHMLAGQNL KFNVEVVAIR EATEEELAHG HVHGAHDHHH DHDHDGGGSG GGSGGGSGGG
YRYRQGGGSG GGSGGGSGGG YRYRQGGGSG GGSGGGSGGG KVAKDLVVSL AYQVRTEDGV
LVDESPVSAP LDYLHGHGSL ISGLETALEG HEVGDKFDVA VGANDAYGQY DENLVQRVPK
DVFMGVDELQ VGMRFLAETD QGPVPVEITA VEDDHVVVDG NHMLAGQNLK FNVEVVAIRE
ATEEELAHGH VHGAHDHHHD HDHDGGGSGG GSGGGSGGGY RYRQGGGSGG GSGGGSGGGY
RYRQGGGSGG GSGGGSGGGT LTVQARQLLS GIVQQQNNEL RAIEAQQHLL QLTVWGTKQL
QARELAVERY LKDQQLLGIW GASGKLIATT AVPWNASWSN KSLEQIWNNM TWMEWDREIN
EYTSLIYSLI EESQNQQEKN EQELLELDKW ASLWNWFNIT NWLWYHGHDH DHDHHHHHH
SEQ ID NO: 9: Six-Helix according to WO2001/044286
MQLLSGIVQQ QNNLLRAIEA QQHLLQLTVW GIKQLQARIL AGGSGGHTTW MEWDREINNY
TSLIHSLIEE SQNQQEKNEQ ELLEGSSGGQ LLSGIVQQQN NLLRAIEAQQ HLLQLTVWGI
KQLQARILAG GSGGHTTWME WDREINNYTS LIHSLIEESQ NQQEKNEQEL LEGSSGGQLL
SGIVQQQNNL LRAIEAQQHL LQLTVWGIKQ LQARILAGGR GGHTTWMEWD REINNYTSLI
HSLIEESQNQ QEKNEQELLG GHHHHHH
SEQ ID NO: 10: HIV gp41 according to WO03/0000877
TLTVQARQLL SGIVQQQNNE LRAIEAQQHL LQLTVWGTKQ LQARELAVER YLKDQQLLGI
WGASGKLIAT TAVPWNASWS NKSLEQIWNN MTWMEWDREI NNYTSLIHSL IEESQNQQEK
NEQELLELDK WASLWNWENI TNWLWY
SEQ ID No: 11: Uniprot P03375 HIV 1 gp41 (pos. 512 to 856)
AVGIGALFLG FLGAAGSTMG AASMTLTVQA RQLLSGIVQQ QNNLLRAIEA QQHLLQLTVW
GIKQLQARIL AVERYLKDQQ LLGIWGCSGK LICTTAVPWN ASWSNKSLEQ IWNNMTWMEW
DREINNYTSL IHSLIEESQN QQEKNEQELL ELDKWASLWN WENIINWLWY IKLFIMIVGG
LVGLRIVFAV LSVVNRVRQG YSPLSFQTHL PIPRGPDRPE GIEEEGGERD RDRSIRLVNG
SLALIWDDLR SLCLFSYHRL RDLLLIVTRI VELLGRRGWE ALKYWWNLLQ YWSQELKNSA
VSLLNATAIA VAEGTDRVIE VVQGAYRAIR HIPRRIRQGL ERILL
SEQ ID NO: 12: Sortase sequence
LPETG
SEQ ID NO: 13: Transglutaminase sequence
YRYRQ
SEQ ID NO: 14: Linker
GGGS
SEQ ID NO: 15: Linker
GGGSGGGSGGGSGGG
SEQ ID NO: 16: Linker
SGGG
SEQ ID NO: 17: His-Tag
HHHHHH
SEQ ID NO: 18: 6helNHR (543-581; 6-Helix N-heptad repeat)
QLLSGIVQQQ NNLLRAIEAQ QHLLQLTVWG IKQLQARIL
SEQ ID NO: 19: 6helCHR (625-662; 6-Helix C-heptad repeat)
HTTWMEWDRE INNYTSLIHS LIEESQNQQE KNEQELLE

### DETAILED DESCRIPTION OF THE INVENTION

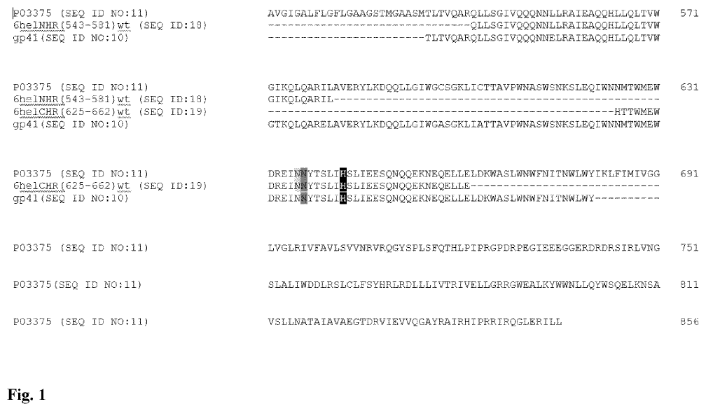

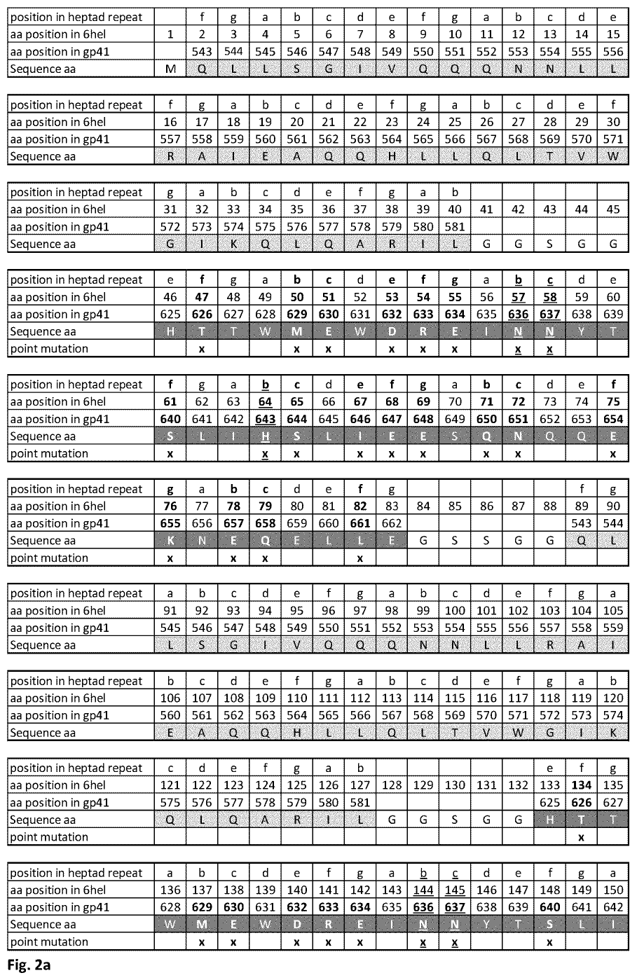

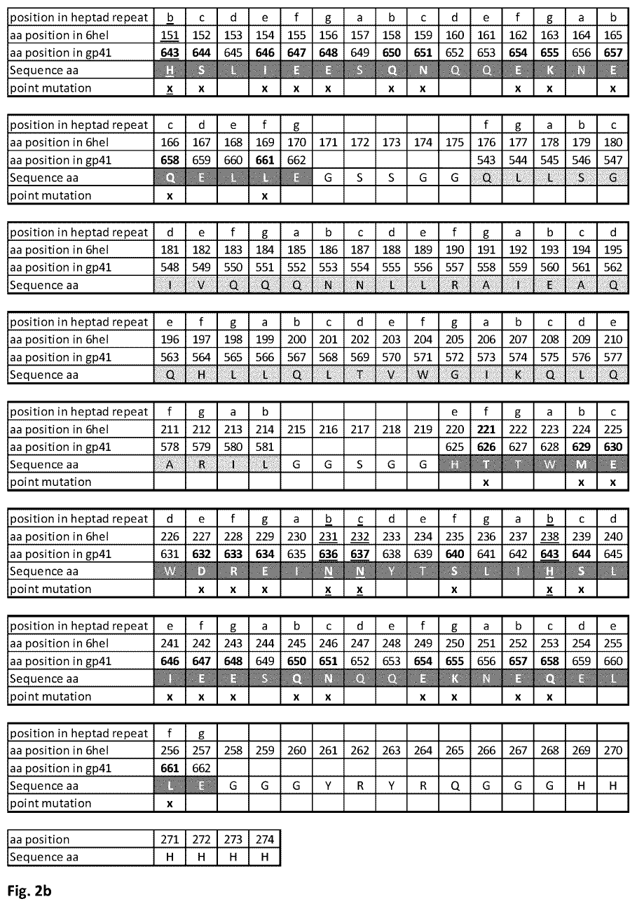

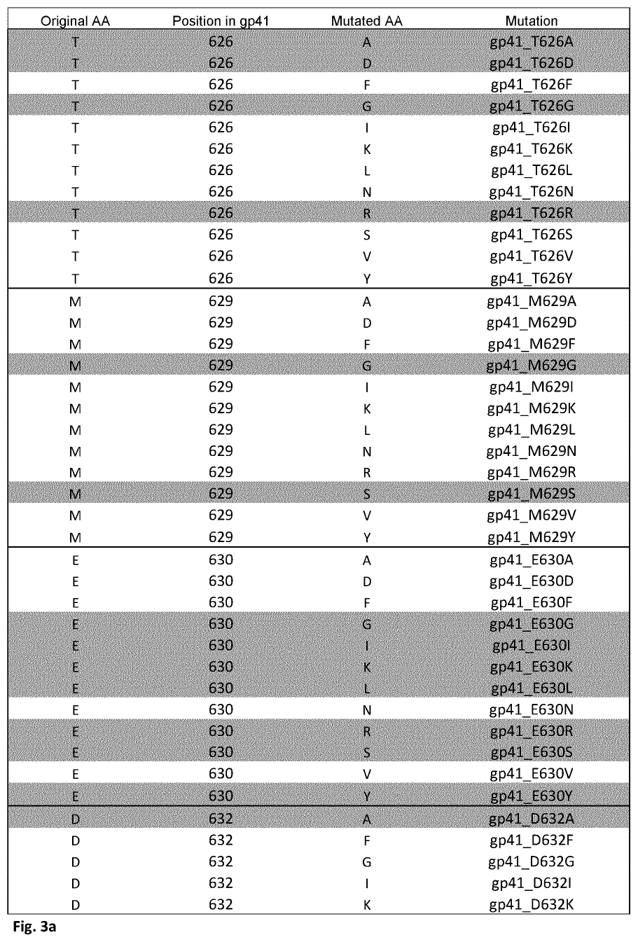

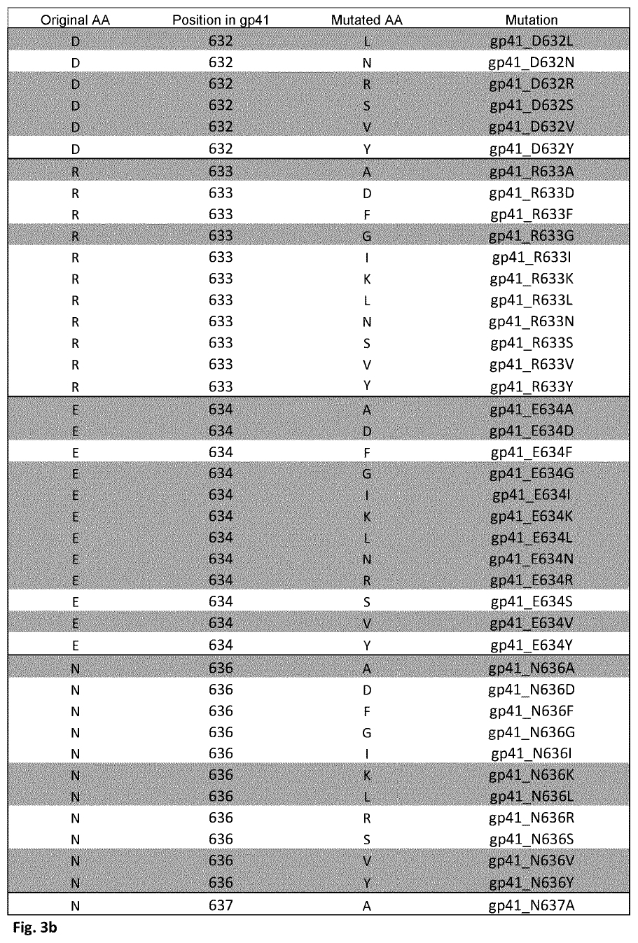

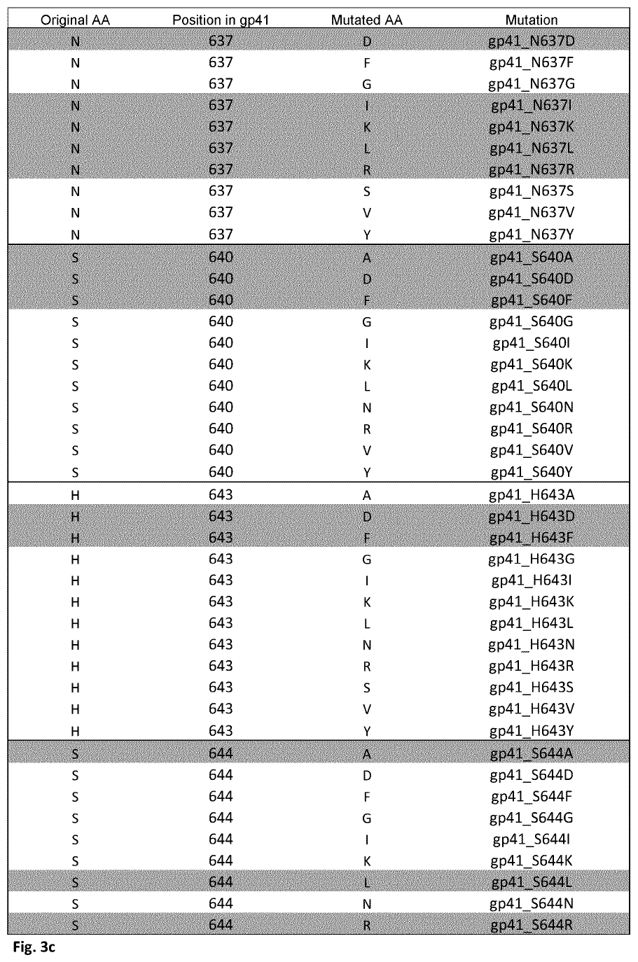

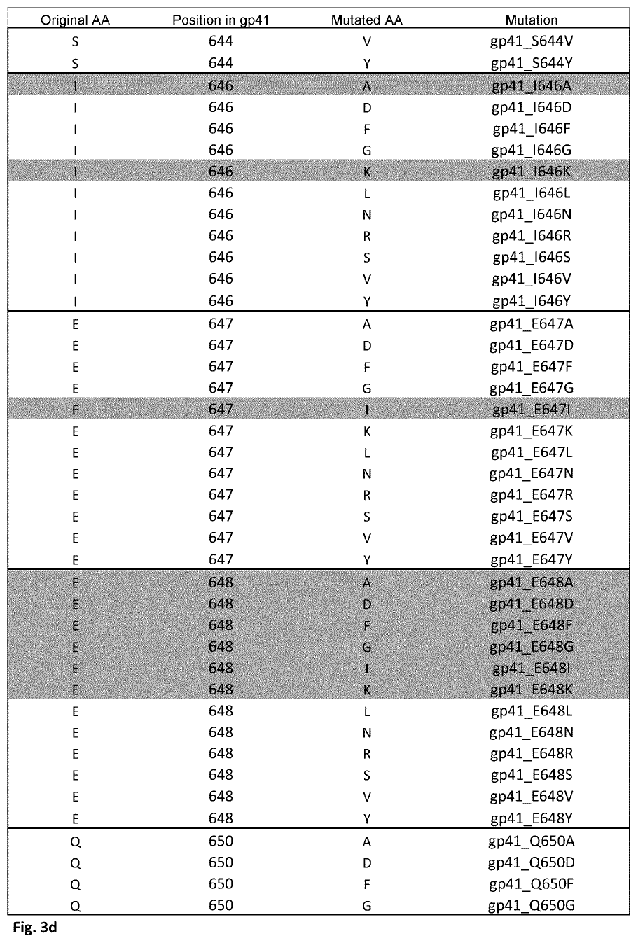

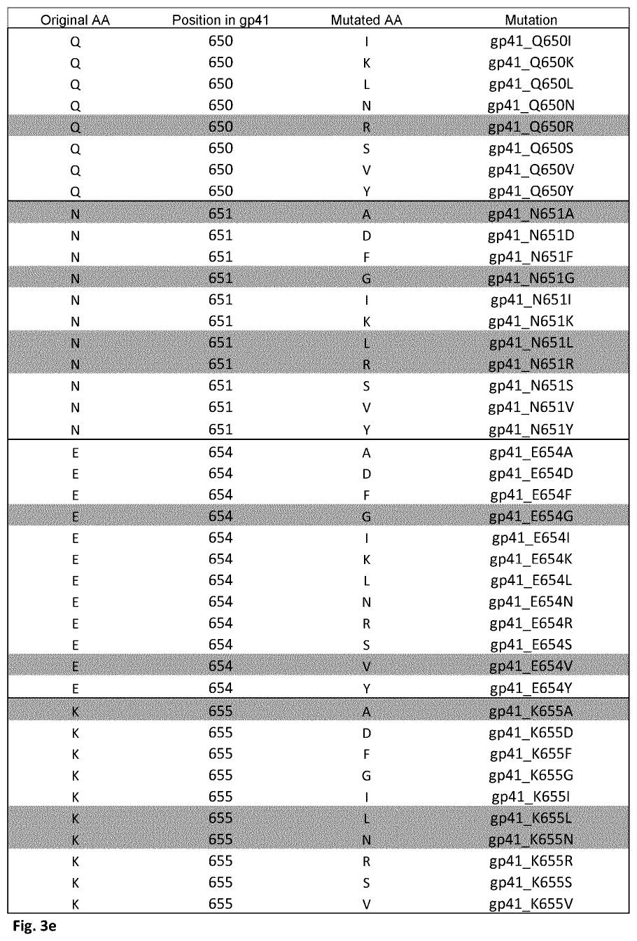

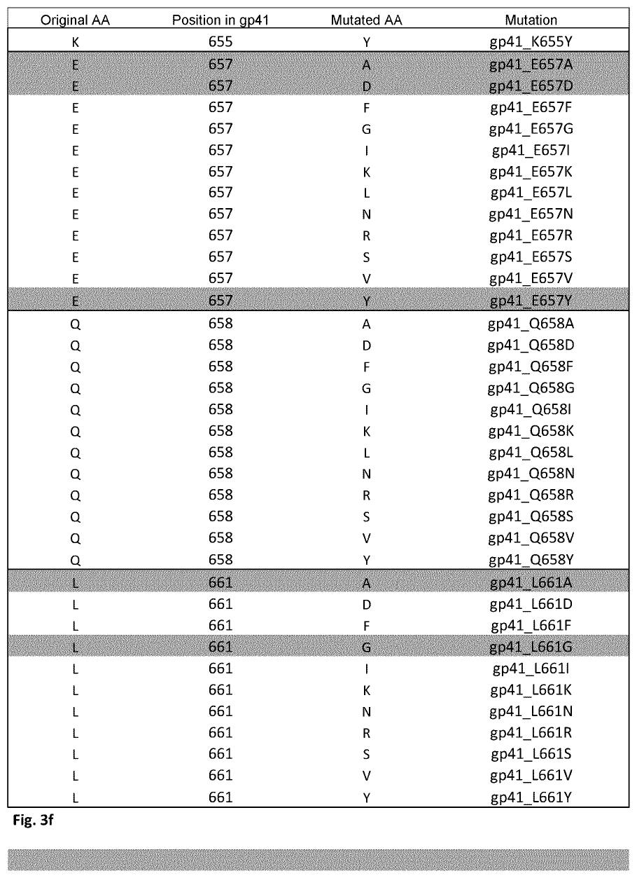

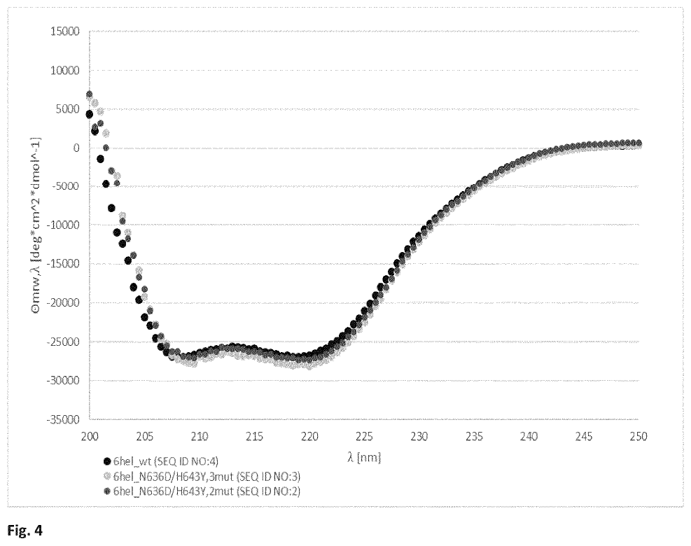

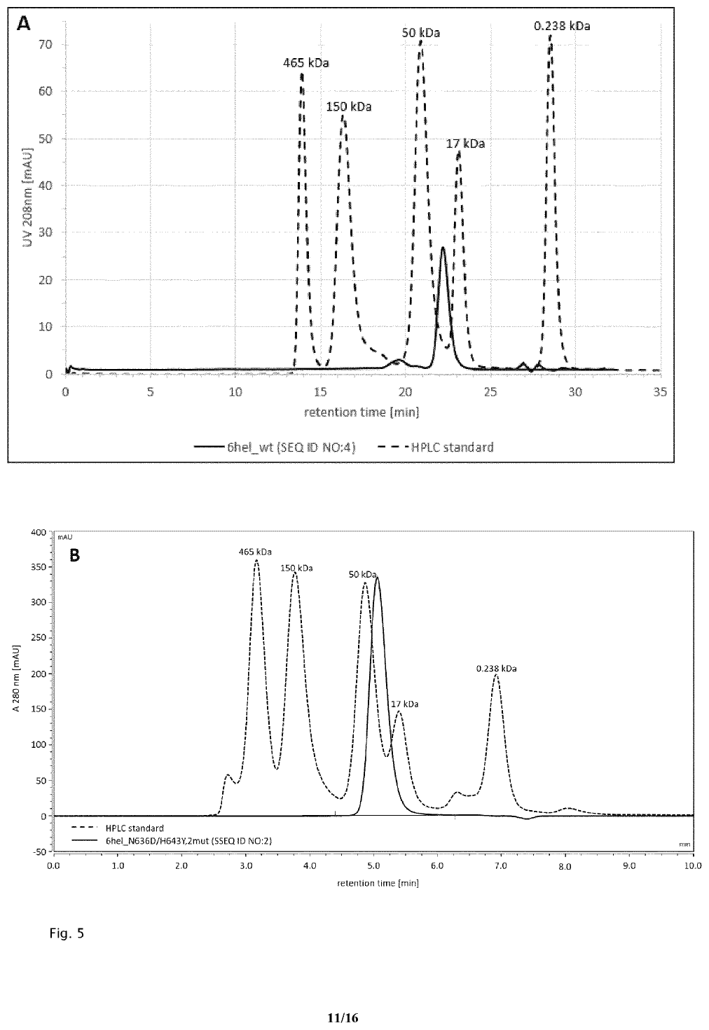

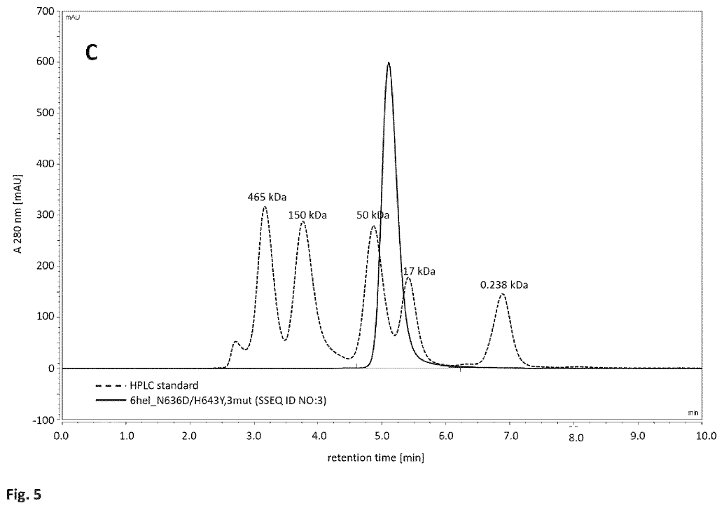

Based on the known structure of the single peptide chain Six-Helix (6hel) construct of gp41 (Root et al., supra) various mutations were introduced into the molecule. As a starting point, positions at the outer side of a single helix that are solvent-exposed, i.e. those being a potential binding site for non-specific antibodies, were mutated by exchanging the original amino acid for a glycine residue. These point mutations did already lead to a specificity improvement in binding gp41 antibodies in a sample. However, the improvement was not finally satisfying. As a next step, the inventors exchanged 21 positions in the C-terminal heptad repeat (CHR, FIG. 1). Each position was exchanged against 12 representative amino acids (arginine, lysine, aspartic acid, serine, asparagine, alanine, valine, isoleucine, phenylalanine, tyrosine and glycine), followed by a small scale expression, purification, modification to design appropriately labeled antigens, and screening for antibody binding. The best variants were then expressed and purified in large scale, labeled and tested. In addition, combinations of point mutations in the Six-Helix (6hel) were introduced, expressed, purified, labeled and also tested for antibody binding. In addition, and as it is known that certain HIV-specific antibodies bind to a particularly immunogenic loop structure that is not part of the Six-Helix, some point mutations were also introduced in the gp41 variant. In total, the inventors of this application designed 242 HIV gp41 mutant antigens (FIG. 3). However, and in contrast to the inventors' expectations, out of these 242 created variants, only very few antigens showed satisfying performance in an immunoassay for detecting suitable gp41 sequences. No regular pattern or consistent logic to identify suitable HIV gp41 variants could be seen.

Surprisingly, out of this large number of variants, the inventors could identify gp41-derived polypeptides and corresponding compositions of peptides that overcome the false positive results in an IVD immunoassay for detecting HIV antibodies to a great extent, thus providing immunological antibody detection with high specificity while maintaining a high sensitivity.

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “HIV gp41” refers to a polypeptide that is derived from the surface protein gp41 of human immunodeficiency virus 1. HIV gp41 mediates both cell attachment and membrane fusion with the host cell of HIV. The wild type sequence can be found under UniProt ID P03375. Positions 535 to 681 of the HIV envelope polyprotein define the gp41 wild type polypeptide. Soluble variants of gp41 have been described e.g. in WO2003/000877.

As used herein, a “patient” means any mammal, fish, reptile or bird that may benefit from the diagnosis, prognosis or treatment described herein. In particular, a “patient” is selected from the group consisting of laboratory animals (e.g. mouse, rat, rabbit, or zebrafish), domestic animals (including e.g. guinea pig, rabbit, horse, donkey, cow, sheep, goat, pig, chicken, camel, cat, dog, turtle, tortoise, snake, lizard or goldfish), or primates including chimpanzees, bonobos, gorillas and human beings. It is particularly preferred that the “patient” is a human being.

The term “sample”, “isolated sample”, “isolated biological sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, urine, saliva, and lymphatic fluid, or solid samples such as tissue extracts, cartilage, bone, synovium, and connective tissue. Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount, concentration or level. The sample is an in vitro sample, isolated from a body, it will be analyzed in vitro and not transferred back into the body.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

For term “sequence comparison” refers to the process wherein one sequence acts as a reference sequence, to which test sequences are compared. When using a sequence comparison algorithm, test and reference sequences are entered into a computer program, if necessary subsequence coordinates are designated, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters. In a sequence alignment, the term “comparison window” refers to those stretches of contiguous positions of a sequence which are compared to a reference stretch of contiguous positions of a sequence having the same number of positions. The number of contiguous positions selected may range from 10 to 1000, i.e. may comprise 20, 30, 40, 50, 60, 70, 80, 90, 100, 150, 200, 250, 300, 350, 400, 450, 500, 550, 600, 650, 700, 750, 800, 850, 900, 950, or 1000 contiguous positions. Typically, the number of contiguous positions ranges from about 20 to 800 contiguous positions, from about 20 to 600 contiguous positions, from about 50 to 400 contiguous positions, from about 50 to about 200 contiguous positions, from about 100 to about 150 contiguous positions. Methods of alignment of sequences for comparison are well known in the art. Optimal alignment of sequences for comparison can be conducted, for example, by the local algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

The term “recombinant DNA molecule” refers to a molecule which is made by the combination of two otherwise separated segments of DNA sequence accomplished by the artificial manipulation of isolated segments of polynucleotides by genetic engineering techniques or by chemical synthesis. In doing so one may join together polynucleotide segments of desired functions to generate a desired combination of functions. Recombinant DNA techniques for expression of proteins in prokaryotic or lower or higher eukaryotic host cells are well known in the art. They have been described e.g. by Sambrook et al., (1989, Molecular Cloning: A Laboratory Manual).

The terms “vector” and “plasmid” are used interchangeably herein, referring to a protein or a polynucleotide or a mixture thereof which is capable of being introduced or of introducing proteins and/or nucleic acids comprised therein into a cell. Examples of plasmids include but are not limited to plasmids, cosmids, phages, viruses or artificial chromosomes.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The term “measurement”, “measuring”, “detecting” or “detection” preferably comprises a qualitative, a semi-quantitative or a quantitative measurement. The term “detecting the presence” refers to a qualitative measurement, indicating the presence of absence without any statement to the quantities (e.g. yes or no statement). The term “detecting amount” refers to a quantitative measurement wherein the absolute number is detected (ng). The term “detecting the concentration” refers to a quantitative measurement wherein the amount is determined in relation to a given volume (e.g. ng/ml).

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (˜2M), CD3, CD4 and CDS.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, γ, δ, ε, and μ. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437).

Typically, in the course of detecting antibodies against HIV antigens in an in vitro diagnostic setting, no differential diagnosis of early IgM antibodies and later stage IgG antibodies is performed.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance based assay (such as the BIAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

The term “antigen (Ag)” is a molecule or molecular structure, which is bound to by an antigen-specific antibody (Ab) or B cell antigen receptor (BCR). The presence of an antigen in the body normally triggers an immune response. In the body, each antibody is specifically produced to match an antigen after cells of the immune system come into contact with it; this allows a precise identification or matching of the antigen and the initiation of a tailored response. In most cases, an antibody can only react to and bind one specific antigen; in some instances, however, antibodies may cross-react and bind more than one antigen. Antigens are normally proteins, peptides (amino acid chains) and polysaccharides (chains of mono-saccharides/simple sugars) or combinations thereof. For the present invention, an antigen is used as a specific ingredient in an immunoassay that specifically binds to antibodies that are present in the analyzed sample and that bind to the antigen. The terms “antigen” and “polypeptide” may be used interchangeably.

In diagnostic tests, antigens are often used in serological test to evaluate if a patient has been exposed to a certain pathogen (e.g. virus or bacterium) and has developed antibodies against such pathogen. Typically, these antigens are produced recombinantly and may be linear peptides or more complex folded molecules aiming to represent native antigens.

To resemble native antigens more closely and to obtain a high epitope density, antigens may be generated by polymerizing monomeric antigens by means of chemical crosslinking. There is a wealth of homobifunctional and heterobifunctional crosslinkers that may be used with great advantage and that are well known in the art. Yet, there are some severe drawbacks in the chemically induced polymerization of antigens for use as specifiers in serological assays. For instance, the insertion of crosslinker moieties into antigens may compromise antigenicity by interfering with the native-like conformation or by masking crucial epitopes. Furthermore, the introduction of non-natural tertiary contacts may interfere with the reversibility of protein folding/unfolding, and it may, additionally, be the source of interference problems which have to be overcome by anti-interference strategies in the immunoassay mixture.

A more recent technique is to fuse the antigen of interest to an oligomeric chaperone, thereby conveying high epitope density to the antigen. The advantage of this technology lies in its high reproducibility and in the triple function of the oligomeric chaperone fusion partner: Firstly, the chaperone enhances the expression rate of the fusion polypeptide in the host cell (e.g. in E. coli), secondly, the chaperone facilitates the refolding process of the target antigen and enhances its overall solubility and, thirdly, it assembles the target antigen reproducibly into an ordered oligomeric structure.

The term “chaperone” is well-known in the art and refers to protein folding helpers which assist the folding and maintenance of the structural integrity of other proteins. Examples of folding helpers are described in detail in WO 2003/000877. Exemplified, chaperones of the peptidyl prolyl isomerase class such as chaperones of the FKBP family can be used for fusion to the antigen variants. Examples of FKBP chaperones suitable as fusion partners are FkpA (aa 26-270, UniProt ID P45523), SlyD (1-165, UniProt ID P0A9K9) and SlpA (2-149, UniProt ID P0AEM0). A further chaperone suitable as a fusion partner is Skp (21-161, UniProt ID P0AEU7), a trimeric chaperone from the periplasm of E. coli, not belonging to the FKBP family. It is not always necessary to use the complete sequence of a chaperone. Functional fragments of chaperones (so-called binding-competent modules) which still possess the required abilities and functions may also be used (cf. WO 98/13496).

The term “comprises no further HIV specific amino acid sequences” means that the HIV gp41 antigen is designed in such a way that antibodies against other HIV antigens like e.g. gp120, p24 or the HIV enzymes protease or reverse transcriptase do not bind to the HIV gp41 antigen. Amino acid sequences derived from other HIV proteins are not part of any of the HIV gp41 antigen. In addition, the term means that no more than 15, in an embodiment no more than 10, in an embodiment no more than 5, in yet another embodiment no more than 2 consecutive amino acids of a known gp41 polypeptide that are part of e.g. UniProt P03375 or SEQ ID NO: 11 are fused to the C- or N-terminal end of an HIV gp41 antigen according to the invention.

Antigens may further comprise an “effector group” such as e.g., a “tag” or a “label”. The term “tag” refers to those effector groups which provide the antigen with the ability to bind to or to be bound to other molecules. Examples of tags include but are not limited to e.g. His tags which are attached to the antigen sequence to allow for its purification. A tag may also include a partner of a bioaffine binding pair which allows the antigen to be bound by the second partner of the binding pair. The term “bioaffine binding pair” refers to two partner molecules (i.e. two partners in one pair) having a strong affinity to bind to each other. Examples of partners of bioaffine binding pairs are a) biotin or biotin analogs/avidin or streptavidin; b) Haptens/anti-hapten antibodies or antibody fragments (e.g. digoxin/anti-digoxin antibodies); c) saccharides/lectins; d) complementary oligonucleotide sequences (e.g. complementary LNA sequences), and in general e) ligands/receptors.

The term “label” refers to those effector groups which allow for the detection of the antigen. Label include but are not limited to spectroscopic, photochemical, biochemical, immunochemical, or chemical, label. Exemplified, suitable labels include fluorescent dyes, luminescent or electrochemiluminescent complexes (e.g. ruthenium or iridium complexes), electron-dense reagents, and enzymatic label.

A “particle” as used herein means a small, localized object to which can be ascribed a physical property such as volume, mass or average size. Particles may accordingly be of a symmetrical, globular, essentially globular or spherical shape, or be of an irregular, asymmetric shape or form. The size of a particle may vary. The term “microparticle” refers to particles with a diameter in the nanometer and micrometer range.

Microparticles as defined herein above may comprise or consist of any suitable material known to the person skilled in the art, e.g. they may comprise or consist of or essentially consist of inorganic or organic material. Typically, they may comprise or consist of or essentially consist of metal or an alloy of metals, or an organic material, or comprise or consist of or essentially consist of carbohydrate elements. Examples of envisaged material for microparticles include agarose, polystyrene, latex, polyvinyl alcohol, silica and ferromagnetic metals, alloys or composition materials. In one embodiment the microparticles are magnetic or ferromagnetic metals, alloys or compositions. In further embodiments, the material may have specific properties and e.g. be hydrophobic, or hydrophilic. Such microparticles typically are dispersed in aqueous solutions and retain a small negative surface charge keeping the microparticles separated and avoiding non-specific clustering.

In one embodiment of the present invention, the microparticles are paramagnetic microparticles and the separation of such particles in the measurement method according to the present disclosure is facilitated by magnetic forces. Magnetic forces are applied to pull the paramagnetic or magnetic particles out of the solution/suspension and to retain them as desired while liquid of the solution/suspension can be removed and the particles can e.g. be washed.

In diagnostic tests, it needs to be decided whether a measured value is classified as “negative” (or “normal” or “non-reactive”) or as “positive” (or “pathologic” or “reactive”). If the measured signal ranges below a predefined threshold, a sample is regarded as nonreactive or negative. If the measured parameter ranges above the threshold, a sample is classified as reactive or positive. Such threshold is a dividing point on a measuring scale that is set for test procedures in order to differentiate between positive and negative values. Said threshold can be selected in such that the test still provides a predefined high sensitivity (high true positive rate) but at the same time also ensures a predefined high specificity (high true negative rate) so that false positive and false negative results are avoided. Depending on the test design and in order to avoid false positive results the cutoff value can be defined as a multiple of the background signal or as a multiple of the result of a normal (negative) sample. Results of tests can be provided in the form of a “cutoff index” (COI) which can be a ratio of a result signal obtained for a sample divided by the predefined cutoff value, resulting in a signal sample/cutoff ratio. In particular in HIV diagnostics, a cutoff and a calculated COI can be chosen in such a way that a high sensitivity and a high specificity of an assay are achieved, i.e. ideally all positives have to be detected and among those positives there should not be any false positives, or at least as few false positives as possible. In many cases, sensitivity and specificity for most highly regulated infectious disease testing is at least 98% (e.g., ranging from 98 to 99.99%). For HIV diagnostics, a minimum sensitivity of 100% and a specificity of >99.8% is required.

A “kit” or “reagent kit” is any manufacture (e.g. a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like. In particular, each of the container means comprises one of the separate elements to be used in the method of the first aspect. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as an optical storage medium (e.g., a Compact Disc) or directly on a computer or data processing device or may be available via a data cloud setup. Moreover, the kit may comprise standard amounts for the biomarkers for calibration purposes.

A “package insert” is used to refer to instructions customarily included in commercial packages of diagnostic products, that contain information about the intended use of the product, instructions how to use the product e.g. on a diagnostic analyser (“Method Sheet”), expected result ranges, interferences observed during development or during the registration process, etc.

### Embodiments

As explained further above, currently available immunoassays for detecting anti-HIV antibodies using gp41-derived antigens show a considerable number of false positive results, i.e. they sometimes lack specificity. Surprisingly, by confining the antigen to the HIV gp41 antigen as explained further below, the number of erroneously reactive samples can be reduced while a high sensitivity of the assay is maintained.

In a first aspect, the present invention relates to a composition suitable for detecting antibodies against HIV gp41 in an isolated sample, said composition comprising at least two, preferably three individual HIV gp41 antigens, wherein a first HIV antigen comprises SEQ ID NO. 1 and wherein the other HIV gp41 antigens comprises at least one of SEQ ID NOs: 2 or and/or 3.

In embodiments, each of said antigens comprises no further HIV specific amino acid sequences.

In embodiments, each of the HIV gp41 antigens is immunoreactive, i.e. antibodies present in a biological sample bind to said antigen. Accordingly, any peptide derived from HIV gp41 which is not bound by antibodies, is not encompassed.

In embodiments, each of the HIV gp41 antigens is soluble and is suitable to be used in in vitro assays aiming to detect antibodies against said antigen in an isolated biological sample.

The composition and each of its HIV gp41 antigens is thus, suitable to be used in in vitro assays aiming to detect anti-HIV antibodies with a high sensitivity and specificity. In embodiments, the sensitivity is >95%, >96%, >97%, >98%, >99%, >99.5%, >99.8%. In particular embodiments, the sensitivity is >99.5% or >99.8%. In particular embodiments, the sensitivity is 100%. In embodiments, the specificity is >95%, >96%, >97%, >98%, >99%, >99.5%. In particular embodiments, the specificity is >99% or >99.5%. In particular embodiments, the specificity is >99.9%. In particular embodiments, the sensitivity is 100% and the specificity is >99.9%.

In embodiments, the composition of HIV gp41 antigens is suitable for detecting or detects antibodies against HIV in a fluid sample. In particular embodiments, the sample is a human sample, in particular in a human body fluid sample. In particular embodiments, the sample is a human blood or urine sample. In particular embodiments the sample is a human whole blood, plasma, or serum sample.

In embodiments, each of the HIV gp41 antigens is in its native state. In particular embodiments, the HIV gp41 specific amino acid sequence comprised in each of the HIV gp41 antigens is folded in its native state.

In embodiments, variants of the HIV gp41 specific amino acid sequences of SEQ ID NOs: 1, 2 and 3 are encompassed. These variants are easily created by a person skilled in the art by conservative or homologous substitutions of the disclosed amino acid sequences (such as e.g. substitutions of a cysteine by alanine or serine). In embodiments, the variant exhibits modifications to its amino acid sequence, in particular selected from the group consisting of amino acid exchanges, deletions or insertions compared to the amino acid sequence of SEQ ID NOs: 1, 2 and 3.

In embodiments, amino acid are C- or N-terminally deleted or inserted at one end or at both ends by 1 to 10 amino acids, in an embodiment by 1 to 5 amino acids. However, no further HIV specific amino acid sequences may be added. In particular, a variant may be an isoform which shows the most prevalent protein isoform. In one embodiment, such a substantially similar protein has a sequence homology to SEQ ID NO: 1, 2 or 3 of at least 95%, in particular of at least 96%, in particular of at least 97%, in particular of at least 98%, in particular of at least 99%.

In embodiments, the variant comprises post-translational modifications, in particular selected from the group consisting of glycosylation or phosphorylation.

It is understood, that such variant classifies as a HIV gp41 antigen variant, i.e. is able to bind and detect anti-HIV gp41 antibodies present in an isolated sample.

In embodiments, the overall three-dimensional structure of each of the HIV gp41 antigens remains unaltered, so that epitopes that were previously (i.e. in the wild type) accessible for binding to antibodies are still accessible in the variant.

In embodiments, at least one of the HIV gp41 antigens further comprises at least one chaperone. Accordingly, the HIV gp41 antigen comprises the HIV gp41 specific amino acid sequences of SEQ ID NO: 1, 2 or 3 as described above or below, and the amino acid sequence of a chaperone.

In a preferred embodiment, only the HIV gp41 antigen of SEQ ID NO: 1 comprises at least one chaperone. In a further preferred embodiment, only the HIV gp41 antigen of SEQ ID NO: 2 comprises at least one chaperone. In a further preferred embodiment, only the HIV gp41 antigen of SEQ ID NO: 3 comprises at least one chaperone.

In embodiments, each of the HIV gp41 antigens further comprises at least one chaperone. Accordingly, the HIV gp41 antigen comprises the HIV gp41 specific amino acid sequences of SEQ ID NO: 1, 2 or 3 as described above or below, and the amino acid sequence of a chaperone.

In particular embodiments, the HIV gp41 antigen comprises two chaperones. In embodiments, said chaperone is selected from the group consisting of SlyD, SlpA, FkpA and Skp. In particular embodiments, the chaperone is SlyD, in particular having an amino acid sequence given in accession no: UniProt ID P0A9K9.

In particular embodiments, the HIV gp41 antigen comprises a HIV gp41 specific amino acid sequence according to SEQ ID NO: 1, 2 or 3 and one SlyD chaperone. In particular embodiments, the HIV gp41 antigen comprises a HIV gp41 specific amino acid sequence according to SEQ ID NO: 1, 2 or 3 and two SlyD chaperones. The fusion of two chaperone results in a higher solubility of the resulting antigen. In particular embodiments, SEQ ID NO: 1 is fused to two SlyD chaperone molecules.

In embodiments, the chaperone is fused to the HIV gp41 specific amino acid sequence at the N- and/or- C-terminus of the HIV gp41 antigen, in particular to the N-terminus of the HIV gp41 antigen. Accordingly, in particular embodiments, the HIV gp41 antigen comprises one SlyD chaperone N-terminally attached to the HIV gp41 specific amino acid sequence. In particular embodiments, the HIV gp41 antigen comprises two SlyD chaperone N-terminally attached to the HIV gp41 specific amino acid sequence. In embodiments, the HIV gp41 antigen comprises one SlyD chaperone N-terminally attached to the HIV gp41 specific amino acid sequence and one SlyD chaperone C-terminally attached to the HIV gp41 specific amino acid sequence.

In embodiments, the HIV gp41 antigen or antigen further comprises linker sequences. These sequences are not specific for anti-HIV gp41 virus antibodies and are not be recognized in an in vitro diagnostic immunoassay. In particular, the HIV gp41 antigen comprises linker sequences between the sequence of the HIV gp41 and the one or more chaperones. In particular embodiments, the linker is a Gly-rich linker. In particular embodiments, the linker has the sequence as indicated in any of SEQ ID NOs: 14, 15 and 16.

In particular embodiments, the HIV gp41 antigen comprises an amino acid sequence according to SEQ ID NO: 5. In embodiments, the HIV gp41 antigen does not comprise any further amino acid sequences. In particular embodiments, the HIV gp41 antigen consists of amino acid sequence according to SEQ ID NO: 5.

In particular embodiments, the HIV gp41 antigen comprises an amino acid sequence according to SEQ ID NO: 6. In embodiments, the HIV gp41 antigen does not comprise any further amino acid sequences. In particular embodiments, the HIV gp41 antigen consists of SEQ ID NO: 6.

In particular embodiments, the HIV gp41 antigen comprises an amino acid sequence according to SEQ ID NO: 7. In embodiments, the HIV gp41 antigen does not comprise any further amino acid sequences. In particular embodiments, the HIV gp41 antigen consists of SEQ ID NO: 7.

In particular embodiments, the HIV gp41 antigen comprises an amino acid sequence according to SEQ ID NO: 8. In embodiments, the HIV gp41 antigen does not comprise any further amino acid sequences. In particular embodiments, the HIV gp41 antigen consists of SEQ ID NO: 8.

It is understood, that an HIV gp41 antigen consisting of SEQ ID NO: 5 or SEQ ID NO: 6 or SEQ ID NO: 7 or SEQ ID NO: 8 does not comprise any additional amino acid sequences, but may still comprise other chemical molecules, such as e.g. labels and/or tags.

In embodiments, the composition suitable for detecting antibodies against HIV gp41 in an isolated sample, comprises at least two, preferably three individual HIV gp41 antigens, wherein a first HIV antigen comprises SEQ ID NO: 1 and wherein a second HIV gp41 antigen comprises at least one of SEQ ID NOs: 2 or 3. In embodiments, said composition comprises HIV gp41 antigens according to SEQ ID NOs: 1, 2 and 3.

In embodiments, said composition comprises SEQ ID NOs: 5, 6, 7 and 8.

In embodiments, each HIV gp41 antigen further comprises a tag or a label. Accordingly, the HIV gp41 antigen comprises the HIV gp41 specific amino acid sequences in any of SEQ ID NO: 1, 2, 3, 6, 7, 8, or 9 as described above or below, and a tag or a label, and optionally the amino acid sequence of one or more chaperones.

In particular embodiments, the tag allows to bind the HIV gp41 antigen directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the HIV gp41 antigen. In particular embodiments, the HIV gp41 specific sequence is labeled. In embodiments, wherein at least one chaperone is present in the antigen, the HIV gp41 specific sequence is labeled or the at least one chaperone is labeled, or both are labeled. In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, the label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In embodiments, the composition comprises one or more additional HIV antigens. In particular embodiments, the composition comprises an HIV gp120 antigen, an HIV reverse transcriptase antigen, or an HIV a p24 antigen or any combination thereof. In particular embodiments, the composition comprises HIV reverse transcriptase antigen as an additional antigen.

In embodiments, the additional HIV antigens are immunoreactive, i.e. antibodies present in a biological sample bind to said antigen. Accordingly, any peptide derived from HIV which is not bound by anti-HIV antibodies, is not encompassed.

In embodiments, the additional HIV gp41 antigen is soluble. The antigen is thus, suitable to be used in in vitro assays aiming to detect antibodies against said antigen in isolated biological sample.

In a second aspect, the present invention relates to a method of producing a composition of HIV gp41 antigens, said method comprising the steps of
a) culturing host cells, in particular E. coli cells, transformed with an expression vector comprising operably linked a recombinant DNA molecule encoding the antigen of the first aspect of the present invention,
b) expression of said antigen, and
c) purification of said antigen, and
d) admixing each of the HIV gp41 antigens obtained by steps a) to c) to form a composition of HIV gp41 antigens.

Optionally, as an additional step e), functional solubilization needs to be carried out so that each HIV gp41 antigen is brought into a soluble and immunoreactive conformation by means of refolding techniques known in the art.

In particular embodiments, the host cells are E. coli cells, CHO cells, or HEK cells. In particular embodiments, the host cells are E. coli cells.

In embodiments, wherein the antigen comprises the HIV gp41 sequence and one or more chaperones, the recombinant DNA molecules according to the invention may also contain sequences encoding linker peptides of 5 to 100 amino acid residues in between the HIV gp41 antigen. Such a linker sequence may for example harbor a proteolytic cleavage site. In an embodiment, the addition of non-HIV gp41-specific linker or peptidic fusion amino acid sequences to the HIV gp41 antigen is possible as these sequences are not specific for anti-HIV antibodies and would not be recognized in an in vitro diagnostic immunoassay.

In a third aspect, the present invention relates to a method for detecting antibodies specific for HIV in an isolated sample, wherein the composition of the first aspect of the present invention, or an HIV gp41 antigen obtained by a method of the second aspect of the present invention is used as a capture reagent and/or as a binding partner for said anti-HIV antibodies.

In a fourth aspect, the present invention relates to a method for detecting antibodies specific for HIV in an isolated sample said method comprising
a) forming an immunoreaction mixture by admixing a body fluid sample with an HIV gp41 antigen composition of the first aspect of the present invention or an HIV gp41 antigen composition obtained by the method of the second aspect of the present invention
b) maintaining said immunoreaction admixture for a time period sufficient for allowing antibodies present in the body fluid sample against said HIV gp41 antigen composition to immunoreact with an HIV gp41 antigen as part of said composition to form an immunoreaction product; and
c) detecting the presence and/or the concentration of any of said immunoreaction product.

In embodiments, the method is an in vitro method. In embodiments, the method exhibits a high sensitivity and specificity. In embodiments, the sensitivity is >95%, >96%, >97%, >98%, >99%, >99.5%. In particular embodiments, the sensitivity is >99% or >99.5%. In particular embodiments, the sensitivity is 100%. In embodiments, the specificity is >95%, >96%, >97%, >98%, >99%, >99.5%. In particular embodiments, the specificity is >99% or >99.5%. In particular embodiments, the specificity is 99.8%. In particular embodiments, the sensitivity is 100% and the specificity is >99.9%.

In embodiments, the antibodies detected by the method of the present invention are anti-HIV virus antibodies of the IgG, the IgM, or the IgA subclass, or of all three subclasses in the same immunoassay.

In embodiments, the antibodies detected are directed against gp41 of the human immunodeficiency virus (HIV), in particular against gp41 of HIV-1.

In embodiments, the isolated biological sample in which the HIV specific antibodies are detected, is a human sample, in particular in a human body fluid sample. In particular embodiments, the sample is a human blood or urine sample. In particular embodiments the sample is a human whole blood, plasma, or serum sample. In particular embodiments the sample is a venous or capillary human whole blood, plasma, or serum sample.

In embodiments, the HIV gp41 antigen admixed to the isolated biological sample in step a) comprises at least one HIV gp41 specific amino acid sequence according to SEQ ID NOs: 1, 2 or 3 or a variant thereof. In embodiments, the HIV gp41 antigen comprises no further HIV gp41 virus specific amino acid sequences.

In embodiments, the composition applied in the method for detecting antibodies specific for HIV in an isolated sample comprises HIV gp41 antigens according to SEQ ID NOs: 5, 6, 7, and 8. In particular embodiments, the HIV specific sequences of said HIV gp41 antigens consist of SEQ ID NOs 5, 6, 7 and 8.

In embodiments, the HIV gp41 antigen is immunoreactive, i.e. antibodies present in a biological sample bind to said antigen. Accordingly, any peptide derived from HIV gp41 which is not bound by antibodies, is not encompassed.

In embodiments, the HIV gp41 antigen is soluble. The HIV gp41 antigen is thus, suitable to be used in in vitro assays aiming to detect antibodies against said antigen in isolated biological sample.

In embodiments, the method comprises the additional step of adding a solid phase to the immunoreaction mixture. In embodiments, the solid phases is a Solid Phase Extraction (SPE) cartridges, or beads. In particular embodiments, the solid phase comprises or consists of particles. In embodiments, the particles are non-magnetic, magnetic, or paramagnetic. In embodiments, the particles are coated. The coating may differ depending on the use intended, i.e. on the intended capture molecule. It is well-known to the skilled person which coating is suitable for which analyte. The particles may be made of various different materials. The beads may have various sizes and comprise a surface with or without pores.

In particular embodiments, the particles are microparticles. In embodiments, the microparticles have a diameter of 50 nanometers to 20 micrometers. In embodiments, the microparticles have a diameter of between 100 nm and 10 μm. In embodiments, the microparticles have a diameter of 200 nm to 5 μm, in particular of 750 nm to 5 μm, in particular of 750 nm to 2 μm. In particular embodiments the microparticles are magnetic or paramagnetic. In particular, the microparticles are paramagnetic.

In embodiments, the solid phase is added either before the addition of the sample to said antigens or after the immunoreaction admixture is formed. Accordingly, the addition of the solid phase may take place in step a) of the present method, in step b) or the present method, or after step b) of the present method.

In embodiments, the performed method is an immunoassay for detecting anti-HIV antibodies in an isolated biological sample. Immunoassays for detection of antibodies are well known in the art, and so are methods for carrying out such assays and practical applications and procedures. The HIV gp41 antigens according to the invention can be used to improve assays for the detection of anti-HIV antibodies independently of the labels used and independently of the mode of detection (e.g., radioisotope assay, enzyme immunoassay, electrochemiluminescence assay, etc.) or the assay principle (e.g., test strip assay, sandwich assay, indirect test concept or homogenous assay, etc.).

In embodiments, the performed method is an immunoassay for detecting anti-HIV antibodies in an isolated sample according to the so-called double antigen sandwich concept (DAGS). Sometimes this assay concept is also termed double antigen bridge concept, because the two antigens are bridged by an antibody analyte. In such an assay the ability of an antibody to bind at least two different molecules of a given antigen with its two (IgG, IgE), four (IgA) or ten (IgM) paratopes is utilized.

In embodiments, an immunoassay for the determination of anti-HIV gp41 antibodies according to the DAGS format is carried out by incubating a sample containing the anti-HIV gp41 antibodies with two different HIV gp41 antigens, i.e. a first (“capture”) HIV gp41 antigen and a second HIV gp41 virus (“detection”) antigen, wherein each of the two antigens is bound specifically by anti-HIV gp41 antibodies.

In embodiments, the structure of the “capture antigen” and the “detection antigen” are immunologically cross-reactive. The essential requirement for performing the present method is that the relevant epitope or the relevant epitopes are present on both antigens. Accordingly, both antigens comprise an HIV gp41 specific amino acid sequence as described above or below. In embodiments, the two antigens comprise the same or different fusion moieties (e.g. SlyD fused to HIV gp41 specific antigen tagged to be bound by a solid phase, and, e.g., FkpA fused to HIV gp41 specific antigen labeled to be detected) as such variations significantly alleviate the problem of non-specific binding and thus mitigate the risk of false-positive results.

In embodiments, the first antigen can be bound directly or indirectly to a solid phase and usually carries an effector group which is part of a bioaffine binding pair. In particular embodiments, the first antigen is conjugated to biotin and the complementary solid phase is coated with either avidin or streptavidin. In embodiments, the second antigen carries a label that confers specific detectability to this antigen molecule, either alone or in complex with other molecules. In particular embodiments, the second antigen carries a ruthenium complex label.

Thus, in step b) of the present method, an immunoreaction admixture is formed comprising the first antigen, the sample antibody and the second antigen.

This ternary complex consisting of analyte antibody sandwiched in between two antigen molecules is termed immunocomplex or immunoreaction product.

In embodiments, the method may comprise the additional step of separating the liquid phase from the solid phase.

Accordingly, in embodiments, the method for detecting antibodies specific for HIV gp41 virus in an isolated sample comprises
a) adding to said sample a first HIV gp41 antigen which can be bound directly or indirectly to a solid phase and carries an effector group which is part of a bioaffine binding pair, and a second HIV gp41 antigen which carries a detectable label, wherein said first and second HIV gp41 antigens bind specifically to said anti-HIV gp41 antibodies
b) forming an immunoreaction admixture comprising the first antigen, the sample antibody and the second antigen wherein a solid phase carrying the corresponding effector group of said bioaffine binding pair is added before, during or after forming the immunoreaction admixture,
c) maintaining said immunoreaction admixture for a time period sufficient for allowing anti-HIV gp41 antibodies against said HIV gp41 antigens in the body fluid sample to immunoreact with said HIV gp41 antigens to form an immunoreaction product,
d) separating the liquid phase from the solid phase
e) detecting the presence of any of said immunoreaction product in the solid or liquid phase or both.

Finally, the presence of any of said immunoreaction product is detected in the solid or liquid phase or both.

In embodiments, the maximal total duration of the method for detecting HIV gp41-antibodies is less than one hour, i.e. less than 60 minutes, in an embodiment less than 30 minutes, in a further embodiment less than 20 minutes, in an embodiment between 15 and 30 minutes, in an embodiment between 15 to 20 minutes. The duration includes pipetting the sample and the reagents necessary to carry out the assay as well as incubation time, optional washing steps, the detection step and also the final output of the result.

In a fifth aspect, the present invention relates to a method of identifying if a patient has been exposed to an HIV infection in the past, comprising
a) forming an immunoreaction mixture by admixing a body fluid sample of the patient with a HIV gp41 antigen composition of the first aspect of the present invention or an HIV gp41 antigen obtained by the method of the second aspect of the present invention
b) maintaining said immunoreaction admixture for a time period sufficient for allowing antibodies present in the body fluid sample against said HIV gp41 antigen composition to immunoreact with an HIV gp41 antigen as part of said HIV gp41 antigen composition to form an immunoreaction product; and
c) detecting the presence and/or absence of any of said immunoreaction product, wherein the presence of an immunoreaction product indicates that the patient has been exposed to an HIV infection in the past.

In embodiments, the patient was exposed to an HIV infection prior to performance of the present method. In particular, the patient was exposed to HIV infection at least 5 days prior to performance of the present method. In particular, the patient was exposed to HIV infection at least 10 days prior to performance of the present method. In particular, the patient was exposed to HIV infection at least 14 days prior to performance of the present method.

In an sixth aspect, the present invention relates to a use of HIV gp41 antigen composition of the first aspect of the present invention or of HIV gp41 antigen composition obtained by the method of the second aspect of the present invention in a high throughput in vitro diagnostic test for the detection of anti-HIV antibodies.

In an seventh aspect, the present invention relates to a reagent kit for the detection of anti-HIV virus antibodies, comprising HIV gp41 antigen composition of the first aspect of the present invention or HIV gp41 antigen composition obtained by the method of the second aspect of the present invention.

In embodiments, the reagent kit comprises in separate containers or in separated compartments of a single container unit, an HIV gp41 antigen composition of the first aspect of the present invention, or the HIV gp41 antigen obtained by the method of the second aspect of the present invention. In particular embodiments, the comprised HIV gp41 antigen is that is covalently coupled to biotin.

In embodiments, the reagent kit further comprises in separate containers or in separated compartments of a single container unit, microparticles, in particular microparticles coated with avidin or streptavidin.

All definitions provided for the HIV gp41 antigens as part of the composition provided for the first to the third aspect of the invention apply mutatis mutandis also for the fourth, fifth, sixth and seventh aspect of the invention.

In further embodiments, the present invention relates to the following items:
Item 1: A composition suitable for detecting antibodies against HIV gp41 in an isolated sample, said composition comprising at least two individual HIV gp41 antigens, wherein a first HIV antigen comprises SEQ ID NO. 1 and wherein a second HIV gp41 antigen comprises SEQ ID NOs: 2 and/or 3, and wherein each of said individual HIV gp41 antigens comprises no further HIV specific amino acid sequences.
Item 2: A composition according to item 1, wherein at least one of said HIV gp41 antigens is fused to at least one chaperone, in an embodiment to two chaperones.
Item 3: A composition according to item 2, wherein said chaperone is selected from the group consisting of SlyD, SlpA, FkpA and Skp.
Item 4: A composition according to item 1 or 2, wherein said chaperone is fused to the HIV gp41 specific amino acid sequence at the N- and/or the C-terminal end of said HIV gp41 antigen.
Item 5: A composition according to any of the preceding items, wherein each of said antigens is soluble and immunoreactive.
Item 6: A composition according to any of the preceding items, wherein said HIV gp41 antigens comprise SEQ ID NOs 1 and 2 or SEQ ID NOs 1 and 3.
Item 7: A composition according to any of the preceding items, wherein said HIV gp41 antigens comprise SEQ ID NOs 1, 2 and 3.
Item 8: A composition according to any of the preceding items, wherein said HIV gp41 antigens comprise SEQ ID NOs 5, 6, 7 and 8.
Item 9: A composition according to any of the preceding items, wherein the HIV specific sequences of said HIV gp41 antigens consist of SEQ ID NOs 5, 6, 7 and 8.
Item 10: An HIV gp41 antigen suitable for detecting antibodies against HIV in an isolated biological sample comprising SEQ ID NO:1,
wherein said antigen comprises no further HIV specific amino acid sequences.
Item 11: An HIV gp41 antigen suitable for detecting antibodies against HIV in an isolated biological sample comprising SEQ ID NO:2,
wherein said antigen comprises no further HIV specific amino acid sequences.
Item 12: An HIV gp41 antigen suitable for detecting antibodies against HIV in an isolated biological sample comprising SEQ ID NO:3, wherein said antigen comprises no further HIV specific amino acid sequences.
Item 13: The HIV gp41 antigen according to any of items 10 to 12, wherein said antigen further comprises a transglutaminase peptide, in an embodiment comprising the amino acid sequence YRYRQ (SEQ ID NO:13).
Item 14: The HIV gp41 antigen according to any of items 10 to 13, wherein said antigen further comprises a sortase peptide, in an embodiment comprising the amino acid sequence LPETG (SEQ ID NO:12).
Item 15: The HIV gp41 antigen according to any of items 10 to 14, wherein said antigen further comprises a linker peptide, in an embodiment comprising two or three glycine residues, in an embodiment GGGS (SEQ ID NO: 14), in another embodiment GGGSGGGSGGGSGGG (SEQ ID NO:15), in another embodiment SGGG (SEQ ID NO:16).
Item 16: The HIV gp41 antigen according to any of items 10 to 15, wherein said antigen further comprises a histidine peptide HHHHHH (SEQ ID NO: 17).
Item 17: A method of producing a composition of HIV gp41 antigens according to any of items 1 to 9, said method comprising for each of said antigens the steps of
a) culturing host cells, transformed with an expression vector comprising operably linked a recombinant DNA molecule encoding one of each of said antigens,
b) expression of each of said antigens,
c) purification of each of said antigens, and
d) admixing an HIV gp41 antigen comprising SEQ ID NO. 1 obtained by steps a) to c) with at least one HIV gp41 antigen comprising at least one of SEQ ID NO: 2 and/or 3 obtained by steps a) to c) to form a composition of HIV gp41 antigens.
Item 18: A method according to item 17, wherein in step d) the admixed HIV gp41 antigens consist of SEQ ID NOs 5, 6, 7 and 8.
Item 19: A method for detecting antibodies specific for HIV gp41 in an isolated sample, wherein a composition of HIV gp41 antigens according to any of items 1 to 9 is used as a capture reagent and/or as a binding partner for said anti-HIV antibodies.
Item 20: A method for detecting antibodies specific for HIV gp41 in an isolated sample, said method comprising
a) forming an immunoreaction mixture by admixing a body fluid sample with an HIV gp41 antigen composition according to any of items 1 to 9,
b) maintaining said immunoreaction admixture for a time period sufficient for allowing antibodies present in the body fluid sample against HIV gp41 to immunoreact with said HIV gp41 antigen composition to form an immunoreaction product; and
c) detecting the presence and/or the concentration of any of said immunoreaction product.
Item 21: A method for detecting antibodies specific for HIV in an isolated sample according to item 20, wherein said immunoreaction is carried out in a double antigen sandwich format comprising
a) adding to said sample a first HIV gp41 antigen according to item 10 to 16 or a first antigen composition according to item 1 to 9 which can be bound directly or indirectly to a solid phase and carries an effector group which is part of a bioaffine binding pair, and a second HIV gp41 antigen according to item 10 to 16 or a second antigen composition according to item 1 to 9 which carries a detectable label, wherein said first and second HIV gp41 antigens bind specifically to said anti-HIV antibodies,
b) forming an immunoreaction admixture comprising the first antigen, the sample antibody and the second antigen wherein a solid phase carrying the corresponding effector group of said bioaffine binding pair is added before, during or after forming the immunoreaction admixture,
c) maintaining said immunoreaction admixture for a time period sufficient for allowing anti-HIV antibodies against said HIV gp41 antigens in the body fluid sample to immunoreact with said HIV gp41 antigens to form an immunoreaction product,
d) separating the liquid phase from the solid phase
e) detecting the presence of any of said immunoreaction product in the solid or liquid phase or both.
Item 22: A method of identifying if a patient has been exposed to an HIV infection in the past, comprising
a) forming an immunoreaction mixture by admixing a body fluid sample of the patient with an HIV gp41 antigen composition according to any of items 1 to 9,
b) maintaining said immunoreaction admixture for a time period sufficient for allowing antibodies present in the body fluid sample against said HIV gp41 antigen composition to immunoreact with said HIV gp41 antigen composition to form an immunoreaction product; and
c) detecting the presence and/or absence of any of said immunoreaction product, wherein the presence of an immunoreaction product indicates that the patient has been exposed to an HIV infection in the past.
Item 23: Use of an HIV gp41 antigen composition according to any of items 1 to 9 for the detection of anti-HIV gp41 antibodies in an isolated sample.
Item 24: Use of an HIV gp41 antigen composition according to item 23 in a high throughput in vitro diagnostic test for the detection of anti-HIV antibodies in an isolated sample.
Item 25: A reagent kit for the detection of anti-HIV antibodies, comprising an HIV gp41 antigen composition according to any of items 1 to 9.
Item 26: A reagent kit for the detection of anti-HIV antibodies, comprising a composition according to any of items 1 to 9, or an HIV gp41 antigen composition obtained by a method according to item 17.
Item 27: A reagent kit according to item 23 comprising in separate containers or in separated compartments of a single container unit at least microparticles coated with avidin or streptavidin, and an HIV gp41 antigen composition according to any of items 1 to 9 or an HIV gp41 antigen composition obtained by a method according to item 17, wherein each of the HIV gp41 antigens is covalently coupled to biotin.
Item 28: A reagent kit according to item 27, comprising in an additional separate container or in an additional separated compartment of a single container an HIV gp41 antigen composition according to any of items 1 to 9 or an HIV gp41 antigen composition obtained by a method according to item 17, wherein each of the HIV gp41 antigens in said additional separate container or additional separated compartment is covalently coupled to a detectable label, in an embodiment to an electrochemiluminescent ruthenium complex.

The following examples and figures are provided to aid the understanding of the present invention, the true scope of which is set forth in the appended claims. It is understood that modifications can be made in the procedures set forth without departing from the spirit of the invention.

### EXAMPLES

### Example 1: Expression and Purification of Recombinant HIV1 gp41 and 6hel Antigens

### Small-Scale Preparation of Recombinant 6Hel Antigens for High Throughput Screening

242 Plasmids containing gp41-6hel genes with different point mutations and a C-terminal hexahistidine-tag were synthesized at Twist Bioscience and cloned into pET29a via the NdeI (5′-end) and XhoI (3′-end) restriction sites.

### Small Scale Expression of Recombinant Gp41-6Hel Protein

After diluting the plasmids in 10 mM Tris-HCl buffer (pH 8.5) to 5 to 10 ng/μl, 1 μl of the DNA was added to HT96 BL21 (DE3) competent cell plates (Novagen). The transformation was done according to the manufacturer protocol and twenty microliter of the transformation reaction plated on 48-well LB-Kanamycin (50 μg/ml) agar plates (Teknova).

The cultivation and expression of all variants was performed in 96-well plate format. For the pre-culture one colony per mutant was picked in 96-well flat bottom micro titer plates (Corning) filled with 200 μl 4×Yeast-Kanamycin (50 μg/ml) medium per well. In addition, each plate contained at least one wild type gp41 antigen as reference. The cells were grown at 37° C. overnight without shaking. For long-term storage 50 μl of 50% (v/v) Glycerol were added before freezing. The expression was done in 96 deep-well plates including 1000 μl 4×Yeast-Kanamycin (50 μg/ml) medium with 0.1 mM IPTG per well. The mutants were expressed at 30° C. and 800 rpm (Microplate Shaker TiMix; Edmund Bühler GmbH) and harvested after 16 h by centrifugation at 4700 rpm for 10 min.

### Small Scale Purification of Recombinant Gp41-6hel Protein

For small scale purification of 6hel antigens, the bacterial cell pellets from 1 ml E. coli culture were lysed with 125 μl 100% BugBuster® (Merck Millipore) according to the manufacturer protocol. After adding 125 μl 2× equilibration buffer (0.1 M NaH₂PO₄ pH 8.0; 1% (v/v) Tween-20; 1 M NaCl; 40 mM Imidazole) and clearance of the cell lysates by centrifugation (4700 rpm, 10 min), the lysates were transferred to 96-well V bottom plates (Corning) using a pipetting robot (Biomek). Small scale purification was carried out with robotic pipet tips called PhyTips (PhyNexus) which are prepacked with Ni-NTA resins. At first, the Phytips were equilibrated with equilibration buffer (0.05 M NaH₂PO₄ pH 8.0; 0.5% (v/v) Tween-20; 0.5 M NaCl; 20 mM Imidazole). Then they were transferred to the samples for protein. binding In order to remove non-specific bound proteins, the Phytips were washed twice with washing buffer 1 (0.05 M NaH₂PO₄ pH 8.0; 0.5% (v/v) Tween-20; 0.5 M NaCl; 20 mM Imidazole), followed by two washing steps with washing buffer 2 (0.05 M NaH₂PO₄ pH 8.0; 0.5% (v/v) Tween-20; 0.15 M NaCl; 20 mM Imidazole). Finally, the 6hel antigens were eluted in 100 μl elution buffer (0.05 M NaH₂PO₄ pH 8.0; 0.5% (v/v) Tween-20; 0.15 M NaCl; 200 mM Imidazole). Protein samples were analyzed by SDS-PAGE gel. After Ni-NTA purification, a buffer exchange to conjugation buffer (0.15 M KH₂PO₄ pH 8.0; 0.1 M KCl; 0.5 mM EDTA) was conducted using Pierce™ 96-well micro-dialysis plates according to the instructions provided by Pierce Biotechnology.

Small Scale Ruthenylation and Biotinylation of Recombinant gp41-6hel Protein

Conjugation was performed in black 96-well half area plates (Corning) using NHS—chemistry. Prior to conjugation protein concentration in each well was determined in micro titer plates by BCA assay using the Pierce™ BCA Protein Assay Kit (ThermoFisher). Per well 180 μl of BCA solution were added to 20 μl of purified antigen and measured at 562 nm with a Tecan Sunrise™ microplate reader. Antigen (approx. 1 mg/ml) and label were rapidly mixed to a final antigen to label ratio of one to four for ruthenium conjugation and one to five for biotin conjugation and a DMSO concentration of 10% (v/v). The plates were incubated at room temperature for 30 min at 600 rpm. The labeling reaction was stopped by adding L-lysine to a final concentration of 10 μM. For small-scale preparations free unbound ruthenium label were not removed, while free unbound biotin label was removed by usage of PD MultiTrap™ G-25 96-well plates (GE Healthcare). Concentrations of ruthenylated and biotinylated antigens were determined by usage of BCA assay as described above. The ruthenylated and biotinylated gp41-6hel mutants were stored at 4° C. until the assessment by the Elecsys test system.

In summary, 171 (71%) out of the 242 6hel mutations could be successfully purified, labeled and further assessed via immunoassay. The missing 71 variants, either failed DNA synthesis, could not be expressed or the yield of purified protein was too low to perform a labeling reaction.

Large-Scale Preparation of Recombinant HIV1 gp41 and 6hel Antigens for Thorough Screening

Plasmids containing recombinant HIV1 gp41 (aa536-681) and 6hel genes with different point mutations and a C-terminal hexahistidine-tag were synthesized at Eurofins Genomics GmbH and cloned into pET24a (+) via the NdeI (5′-end) and XhoI (3′-end) restriction sites.

Furthermore, recombinant gp41 (aa536-681) was N-terminally fused to two SlyD chaperones from E. coli via a Glycine-Serine rich linker (Scholz, C. et al., J. Mol. Biol. (2005) 345, 1229-1241) resulting in the EcSlyD-EcSlyD-gp41 fusion protein in the following only referred to as gp41.

Expression of gp41 as well as 6hel constructs was performed in BLR(DE3) E. coli cells using standard LB medium and IPTG induction for three hours at 37° C. Cells were harvested by centrifugation (20 min, 5000 g) and stored at −20° C. upon further processing.

Large Scale Purification of Recombinant HIV1 gp41 and 6hel Antigens

Recombinant HIV1 gp41 and 6hel antigens were purified under denaturing conditions followed by an on-column renaturation. In detail, bacterial pellets from 700 ml E. coli culture were resuspended in chaotropic lysis buffer (50 mM sodium phosphate pH 8.0; 4 M guanidinium chloride; 5 mM imidazole) and stirred at room temperature for 90 min. For clearance the cell lysate was centrifuged and filtered (5/0.8/0.2 μm). Clarified supernatant was applied to a Roche complete His-tag purification column equilibrated with lysis-buffer. Unspecifically bound proteins were removed from the column by a thorough wash with lysis-buffer to baseline. Refolding of antigens was performed by on-column renaturation using refolding-buffer (50 mM sodium phosphate pH 8.0; 100 mM NaCl). Refolded target protein was eluted from the column with imidazole containing elution buffer (50 mM sodium phosphate pH 8.0; 50 mM imidazole; 100 mM NaCl). For buffer exchange and polishing, the protein was applied to a Superdex 200 column equilibrated with SEC-buffer1 (50 mM Tris-HCl pH 8.0; 150 mM KCl) for site specific labeling or SEC-buffer2 (150 mM potassium phosphate pH 8.9; 100 mM KCl; 0.5 mM EDTA) for labeling using NHS-chemistry. Gp41 elutes in three peaks with one prominent peak representing an oligomeric arrangement. The oligomeric fraction was concentrated and processed to biotinylation and ruthenylation. 6hel elutes in one peak, which was concentrated and processed to biotinylation and ruthenylation

### Example 2: Ruthenylation and Biotinylation of Recombinant Gp41 Protein

Large Scale Ruthenylation and Biotinylation of Recombinant HIV1 gp41 and 6hel Antigens Using NHS-Chemistry

For conjugation of the antigens with Biotin or Ruthenium protein concentration should be ideally 10 mg/ml in SEC-buffer 2. Conjugation was performed with a molar antigen to label ratio of one to four and a DMSO concentration of 5% (v/v) using NSH-chemistry. Label and antigen were rapidly mixed and stirred at room temperature for 30 min. The labeling reaction was stopped by adding L-lysine to a final concentration of 10 mM. For large scale preparations free unbound label was removed from the reaction by size exclusion chromatography using a Superdex 200 Increase (GE Healthcare) column equilibrated with storage-buffer (50 mM sodium phosphate pH 7.5; 100 mM KCl; 0.5 mM EDTA). Concentration of ruthenylated antigens was determined by the usage of BCA assay and concentration of biotinylated antigens was done by absorption measurement at 280 nm.

Large Scale Ruthenylation and Biotinylation of Recombinant HIV1 gp41 and 6hel Using Transglutaminase

Recombinant Transglutaminase from Kutzneria albida (KalbTG) can be used to site specifically label antigens by forming a Gln-Lys isopeptide bond between the Q-tag containing antigen and the respective containing K-tag label (Steffen, W. et al. J. Mol. Biol. (2017) 292, 15622-1563). For conjugation of the HIV1 antigens with Biotin or Ruthenium the protein concentration should be ideally 10 mg/ml in SEC-buffer2. Conjugation was performed with a molar Q-tag to label ratio of 1:5 and an enzyme to antigen dearth of 1:300. Antigen, label and activated enzyme were mixed and incubated for 20 hours at 37° C. while gentle mixing. After 20 hours of incubation, the reaction was stopped by adding 10 mM ammonium sulfate. Finally, free unbound label and KalbTG was removed from the labeled antigen by size exclusion chromatography using a Superdex 200 Increase (GE Healthcare) column equilibrated with storage-buffer (50 mM sodium phosphate pH 7.5; 100 mM KCl; 0.5 mM EDTA). Concentration of ruthenylated antigens was determined by the usage of BCA assay and concentration of biotinylated antigens was determined by absorption measurement at 280 nm.

Large Scale Biotinylation of Recombinant HIV1 gp41 and 6hel Using Sortase

Recombinant sortase can be used to site specifically label antigens by forming a peptide bond between the threonine of the C-terminal sortase recognition site (LPETG) and a glycine residue in the respective label. For conjugation of the HIV1 antigens with Biotin by sortase the protein concentration should be ideally, 10 mg/ml in phosphate free SEC-buffer1. Conjugation was performed in the presence of 10 mM calcium chloride with an antigen to label ratio of 1:50 and an enzyme input of 50 U per μmol antigen. Antigen, label and activated enzyme were mixed and incubated for 1 hour at 37° C. while gentle mixing. After 1 hours of incubation, the reaction was loaded on Roche complete His-tag resin to remove sortase as well as unlabeled antigen from the reaction mix. Finally, free unbound label was removed by size exclusion chromatography using a Superdex 200 Increase (GE Healthcare) column equilibrated with storage-buffer (50 mM sodium phosphate pH 7.5; 100 mM KCl; 0.5 mM EDTA). Concentration of biotinylated antigens was determined by absorption measurement at 280 nm.

### Example 3: Biochemical Analysis of Recombinant HIV1 gp41 and 6hel Antigens

Spectroscopic Measurements of Recombinant HIV1 gp41 and 6hel Antigens

Protein concentration measurements were performed with a NanoDrop One® Micro-UV/Vis-spectrophotometer (Thermo Scientific). The molar extinction coefficients (E280 nm) of the antigens was calculated using the equation reported in Pace et al. (Protein Sci. 1995 November; 4 (11): 2411-23).

TABLE 1
Protein parameters of the five best recombinant
HIV1 gp41 and 6hel antigens

| | number | molecular | | ε_{280 nm} |
|---|---|---|---|---|
| | of aa | weight | | [M− |
| antigen variant | residues | [Da] | pI | 1cm−1] |
| 6hel-N(543-581)x3/C(625- | 269 | 30635.14 | 5.46 | 53970 |
| 662)x3-Srt (SEQ ID NO: 4) | | | | |
| 6hel-N(543-581)x3/C(625- | 275 | 31064.47 | 5.03 | 56950 |
| 662, N636D/H643Y)x2/C(625- | | | | |
| 662)x1-Srt (SEQ ID NO: 5) | | | | |
| 6hel-N(543-581)x3/C(625- | 274 | 31273.72 | 5.10 | 61420 |
| 662, N636D/H643Y)x3-Qtag | | | | |
| (SEQ ID NO: 6) | | | | |
| EcSlyD-EcSlyD-gp41((536- | 547 | 59100.63 | 4.84 | 86860 |
| 681; N637E/H643Y)-Q-tag | | | | |
| (SEQ ID NO: 7) | | | | |
| EcSlyD-EcSlyD-Qtag-gp41((536- | 599 | 63722.31 | 5.01 | 95800 |
| 681; N637E/H643Y) | | | | |
| (SEQ ID NO: 8) | | | | |

### Circular Dichroism (CD) Spectra of Recombinant HIV1 6hel Antigens

Far-UV CD spectra (190-250 nm) of 6hel antigens were recorded with a Jasco-720 spectropolarimeter and finally converted into the mean residue ellipticity (θ_{mrw,λ}). All samples were diluted to 0.21 mg/ml in 50 mM potassium phosphate pH 7.5, 100 mM KCl, 0.5 mM EDTA. Adjustments at the spectrometer during measurement were as follow: 0.2 cm pathlength, scanning range of 190-330 nm, with a scanning speed of 20 nm/min, a bandwidth of 2.0 nm, a resolution of 0.5 nm and a response of 1 sec. All spectra were measured in nine repeats and averaged.

In the far-UV range CD spectroscopy allows to analyze the secondary structure proteins as absorption in this UV range is mainly caused by the peptide bond. Therefore, far-UV CD spectra of the all-helical 6hel antigen in comparison to the mutated variants give reliable insights into the structure of the antigen and the effect of the point mutations on protein folding.

### HPLC Analysis of Recombinant HIV1 6hel Antigens

To analyse the purity and the aggregation tendency of the mutated antigens and also to estimate the molecular weight of the purified 6hel antigens HPLC analysis was performed. Therefore, at least 25 μg of the recombinant proteins was loaded onto a Superdex 200 column using 50 mM potassium phosphate pH 7.5, 100 mM KCl and 0.5 mM EDTA as mobile phase. As a reference, an internal HPLC standard was analyzed too. The HPLC analysis allows to assess the aggregation behavior of the mutated 6hel antigens in comparison to the wild type construct.

### Example 4: Immunological Reactivity of the Different Recombinant HIV1 gp41 and 6hel Antigens in an Anti-HIV Immunoassay

The immunological reactivity (antigenicity) of the HIV1 gp41 and 6hel variants was assessed in automated Elecsys® cobas analyzers (Roche Diagnostics GmbH) using the double antigen sandwich (DAGS) format. Signal detection in automated Elecsys® cobas analyzers is based on electrochemiluminescence. In case of a DAGS assay format the biotinylated capture-antigen is immobilized on the surface of a streptavidin coated magnetic bead whereas the same detection-antigen is conjugated with a ruthenium complex. Upon activation the ruthenium complex switches between the redox states 2+ and 3+ resulting in a light signal. In the presence of specific immunoglobulins, in this case anti-HIV IgG antibodies in human sera, the ruthenium complex is bridged to the solid phase and light emission at 620 nm is triggered at the electrode by adding tripropylamine.

All 171 mutated variants of recombinant 6hel from small scale expression and labeling (FIG. 3) were examined in this study to evaluate their binding potential to anti-HIV1 IgG antibodies.

Immunological Reactivity of the Different Recombinant HIV1 gp41 and 6hel Antigens in a DAGS Assay Setup

For a more thorough analysis approximately the best 20 mutations in the 6hel antigen, which were identified in an initial screening to have an improved immunological specificity, were expressed and labeled in large scale and comprehensively analyzed in a DAGS assay setup. Furthermore, the same selected mutations were also transferred into the HIV1 gp41 antigen (WO03/000877) and their specificity evaluated. In addition to these constructs, 6hel as well as gp41 antigens containing combinations of the most promising mutations were produced and assessed.

In detail, the different gp41-biotin or 6hel-biotin and gp41-ruthenium or 6hel-ruthenium antigens were used in reagent buffer 1 (R1) and R2, respectively. Labeled recombinant gp41 antigens were used at concentrations between 30 ng/ml and 300 ng/ml in R1 and R2. The concentration of the various labeled 6hel antigens was between 2 ng/ml and 130 ng/ml in R1 and R2 dependent on the mutation.

To avoid immunological cross reactions via the chaperone fusion units of the recombinant HIV1 gp41 antigens unlabeled EcSkp-EcSlyD-EcSlyD (EP2893021 (B1)) or chemically polymerized EcSlyD-EcSlyD were added in large excess (5-30 μg/ml) to the reaction buffer as anti-interference substances. To assess the specificity and the sensitivity of the different recombinant HIV1 gp41 and 6hel antigens Elecsys measurements with HIV negative and positive as well as seroconversion samples were analyzed.

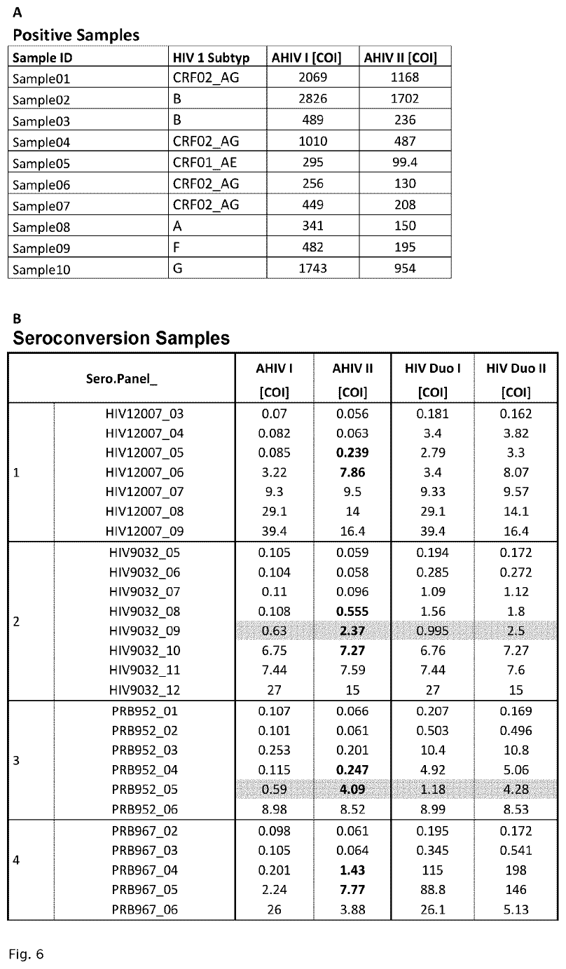

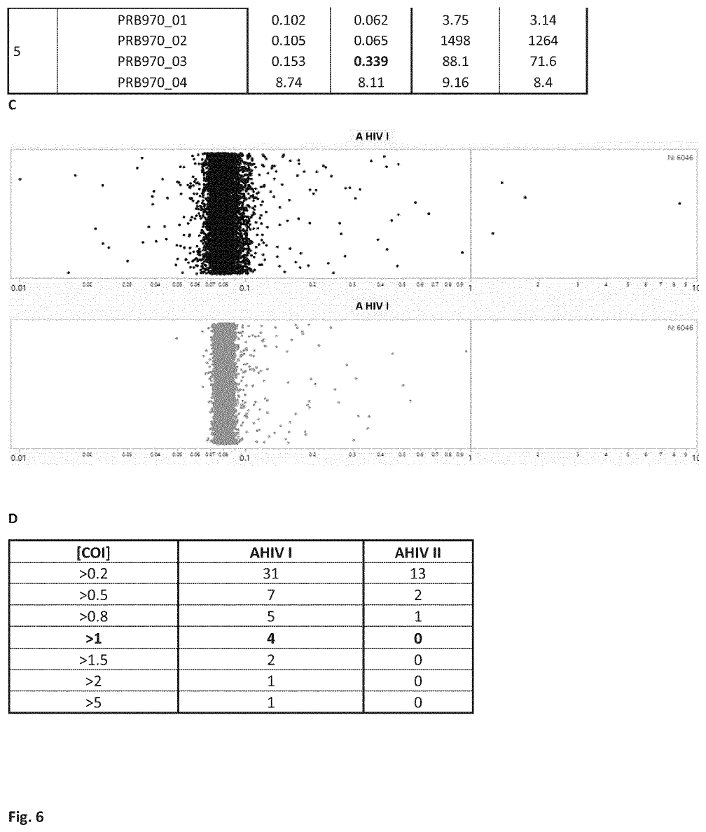

The results of three of the best antigens (SEQ ID Nos 1, 2 and 3) are shown in FIG. 6. In detail, A) Cut-off indices (COI) of ten highly positives HIV samples from patients infected with different HIV-1 subtypes. All samples tested with the improved anti-HIV module (AHIVII) are positive as already with the standard AHIVI module. COI values<1 are indicated as non-reactive, whereas samples with a COI>1 indicate the presence of anti-HIV antibodies. B) Comparison of the performance of the standard Elecsys HIV Duo assay (HIV Duo I in black) with the optimized Elecsys HIV Duo II assay (HIV Duo II in grey), as well as the separate comparison of the anti-HIV module AHIV I and AHIV II in grey and black, respectively. The comparison was performed on five commercially available seroconversion panels (1-5) with sequential blood draws. The optimized anti-HIV II module shows a higher sensitivity compared to the AHIV I module. The higher sensitivity of the AHIV II module particularly kicks in in the seroconversion panel two and three. In these two panels, blood draw nine or five (highlighted in grey) are negative in the AHIV I module and become clearly positive in the optimized AHIV II module. This higher sensitivity reduces the risk of the second window phase after infection and significantly decreases the risk of false negative HIV results. C) Graphical and D) Numerical representation of the specificity of the present and the optimized anti-HIV module in black and grey, respectively. Determination of the specificity of both modules was performed with 6046 HIV negative routine samples from different vendors. Setting the threshold of HIV positivity to a COI>1 (highlighted in bold) the standard AHIV I module showed four false positive samples resulting in a specificity of 99.92. Whereas the optimized AHIV II module does not show any signals with a COI>1, resulting in a specificity of 100. In summary, the here performed experiments showed a significant optimization in sensitivity as well as specificity of the AHIV II module compared to the AHIV module.

### Example 5: Data of an External Specificity Study

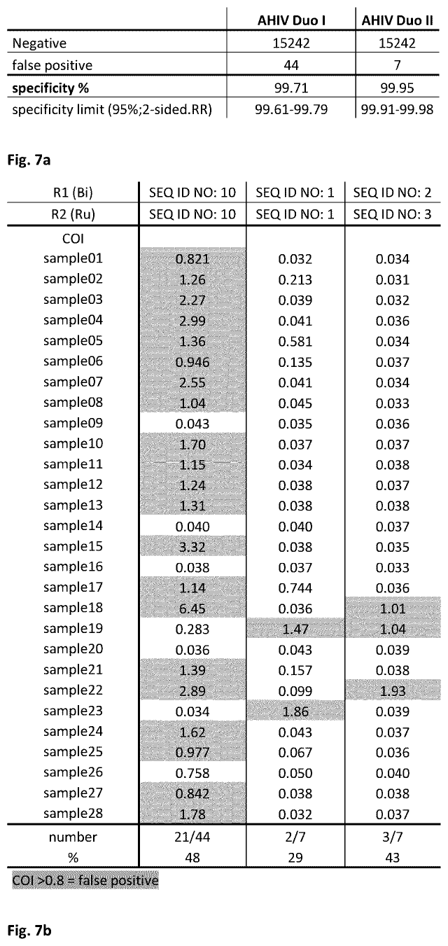

To thoroughly evaluate the specificity of the mutated and thus optimized gp41 and 6hel antigens 15,242 routine blood samples were analyzed in an external study (FIG. 7a). The assessment was performed by an independent laboratory using both the AHIV module of the Elecsys HIV Duo as well of the optimized Elecsys HIV Duo II assay (containing SEQ ID NOs 1, 2 and 3).

Within this study 44 samples resulted in false positive signals within the Elecsys HIV Duo I assay (specificity 99.71%) whereas only 7 false positive samples were detected using the optimized HIV Duo II assay (specificity 99.95%) (FIG. 7A). 21 false positive samples of the Elecsys HIV Duo I assay and 4 of the HIV Duo II are caused by the gp41 and 6hel antigens within the anti-HIV module of the HIV Duo Elecsys assays (FIG. 7b). Thus the significant specificity improvement of the mutated and optimized gp41 and 6hel antigens could also be shown within an independent external study and the interference potential of the gp41 antigen dropped to <20% after optimization.

### Example 6: Improvement of Sensitivity and Specificity of an HIV Immunoassay by Combinations of Mutated and Optimized HIV Gp41 Antigens

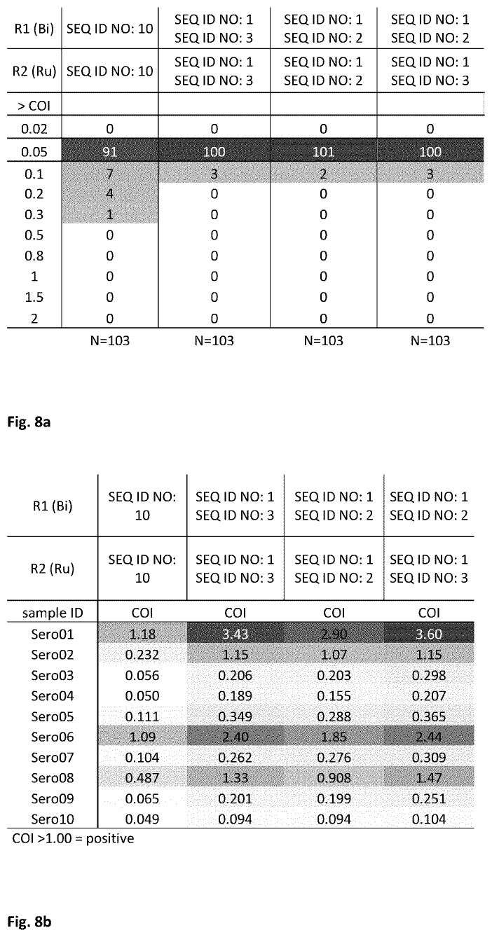

In comparison to an HIV immunoassay containing HIV gp41 antigens comprising SEQ ID NO. 10, not only a combination of SEQ ID NO. 1, 2 and 3 show a significantly improved antigenicity but already a combination of two of the optimized HIV gp41 antigens (SEQ ID NO. 1 and 2 or SEQ ID NO. 1 and 3) show a significantly improved immunological reactivity (FIG. 8). The specificity of the different antigen combinations was assessed by the analysis of 103 HIV negative samples (FIG. 8a). When using the not further optimized gp41 antigen only 91 out of the 103 negative samples (88.35%) show a COI between 0.02 and 0.05, whereas at least 98% of the negative samples are within this COI range when using different combinations of the mutated gp41 and 6hel antigens (FIG. 8a column 2 to 3). Thus the scattering of the samples is significantly lower when using the optimized antigens. In addition, not only the specificity and the scattering is improved but also the sensitivity of the different combined optimized gp41 and 6hel antigens is significantly better (FIG. 8b). For examples sample Sero01 clearly shows that the sensitivity is much higher in all combinations using the mutated antigens, with being SEQ ID NO. 1, 2 and 3 the best combination.

1. A composition suitable for detecting antibodies against HIV gp41 in an isolated sample, said composition comprising at least two individual HIV gp41 antigens, wherein a first HIV antigen comprises SEQ ID NO. 1 and wherein a second HIV gp41 antigen comprises SEQ ID NOs: 2 and/or 3, and wherein each of said individual HIV gp41 antigens comprises no further HIV specific amino acid sequences.

2. A composition according to claim 1, wherein at least one of said HIV gp41 antigens is fused to at least one chaperone.

3. A composition according to claim 2, wherein said chaperone is selected from the group consisting of SlyD, SlpA, FkpA, and Skp.

4. A composition according to claim 1, wherein each of said antigens is soluble and immunoreactive.

5. A composition according to claim 1, wherein said HIV gp41 antigens comprise SEQ ID NOs: 1 and 2 or SEQ ID NOs; 1 and 3.

6. A composition according to claim 1, wherein said HIV gp41 antigens comprise SEQ ID NO: 1, SEQ ID NO: 2, and SEQ ID NO: 3.

7. A method of producing a composition of HIV gp41 antigens according to claim 1, said method comprising for each of said antigens the steps of
a) culturing host cells, transformed with an expression vector comprising operably linked a recombinant DNA molecule encoding one of each of said antigens;
b) expression of each of said antigens;
c) purification of each of said antigens; and
d) admixing an HIV gp41 antigen comprising SEQ ID NO. 1 obtained by steps a) to c) with at least one HIV gp41 antigen comprising at least one of SEQ ID NO: 2 or 3 obtained by steps a) to c) to form a composition of HIV gp41 antigens.

8. A method for detecting antibodies specific for HIV gp41 in an isolated sample, wherein a composition of HIV gp41 antigens according to claim 1 is used as a capture reagent and/or as a binding partner for said anti-HIV antibodies.

9. A method for detecting antibodies specific for HIV gp41 in an isolated sample, said method comprising:
a) forming an immunoreaction mixture by admixing a body fluid sample with an HIV gp41 antigen composition according to claim 1;
b) maintaining said immunoreaction admixture for a time period sufficient for allowing antibodies present in the body fluid sample against HIV gp41 to immunoreact with an HIV gp41 antigen as part of said HIV gp41 antigen composition to form an immunoreaction product; and
c) detecting the presence and/or the concentration of any of said immunoreaction product.

10. A method of identifying if a patient has been exposed to an HIV infection in the past, comprising:
a) forming an immunoreaction mixture by admixing a body fluid sample of the patient with an HIV gp41 antigen composition according to claim 1;
b) maintaining said immunoreaction admixture for a time period sufficient for allowing antibodies present in the body fluid sample against said HIV gp41 antigen composition to immunoreact with an HIV gp41 antigen as part of said HIV gp41 antigen composition to form an immunoreaction product; and
c) detecting the presence and/or absence of any of said immunoreaction product, wherein the presence of an immunoreaction product indicates that the patient has been exposed to an HIV infection in the past.

11. (canceled)

12. A reagent kit for the detection of anti-HIV antibodies, comprising an HIV gp41 antigen composition according to claim 1.

13. A reagent kit according to claim 12, comprising in separate containers or in separated compartments of a single container unit at least microparticles coated with avidin or streptavidin, and an HIV gp41 antigen composition, wherein each of the individual HIV gp41 antigens is covalently coupled to biotin.

14. A reagent kit according to claim 13, further comprising in an additional separate container or in an additional separated compartment of a single container an HIV gp41 antigen composition, wherein each of the individual HIV gp41 antigens in said additional separate container or additional separated compartment is covalently coupled to a detectable label.
